(19) United States
(12) Patent Application Publication
(10) Pub. No.: US 2023/0285256 A1
(43) Pub. Date: Sep. 14, 2023

(54) COMPOSITIONS AND METHODS FOR CURLY HAIR

(71) Applicant: L'Oreal, Paris (FR)

(72) Inventors: Ronak RUGHANI, Edison, NJ (US); Cho-Cho KHINE, Bridgewater, NJ (US); Priveledge MAZONDE, Clark, NJ (US); Kazumitsu KAWAKAMI, Clark, NJ (US)

(21) Appl. No.: 18/115,247

(22) Filed: Feb. 28, 2023

Related U.S. Application Data
(60) Provisional application No. 63/315,044, filed on Feb. 28, 2022.

(30) Foreign Application Priority Data
Apr. 26, 2022 (FR) 2203845

(51) Int. Cl. A61K 8/362; A61K 8/42; A61Q 5/06

(52) U.S. Cl. CPC A61K 8/362; A61K 8/42; A61Q 5/06; A61K 2800/34; A61K 2800/592

(57) ABSTRACT

The disclosure relates to compositions for treating, caring for, and/or styling hair, such as for improving curl definition, regularity, and/or elongation of curly hair. The compositions comprise (a) a synergistic combination of citric acid and at least one urea compound chosen from urea and/or derivatives thereof; (b) at least one additional component for styling and/or caring for hair; and (c) at least one solvent. The disclosure also relates to kits comprising the compositions and methods of using the compositions.

### CROSS REFERENCE TO RELATED APPLICATIONS

This application claims priority to U.S. Provisional Application No. 63/315,044, filed on Feb. 28, 2022, and French Application No. 2203845, filed on Apr. 26, 2022, both of which are incorporated by reference herein in their entireties.

### FIELD OF THE DISCLOSURE

The present disclosure relates to compositions for treating, caring for, and styling hair, as well as packaging systems and kits comprising the compositions, and methods of using the compositions.

### BACKGROUND

Many people are dissatisfied with the appearance of their hair, and desire to treat their hair in order to improve the appearance and/or feel thereof. Consumers primarily use hair care products formulated for their hair texture and style and expect to achieve their desired result.

For instance, curly hair consumers may have concerns that their curly hair appears shorter than its actual length (shrinkage), and/or that the curls are not sufficiently defined. People having curly hair may desire to maintain the curls and may also desire to gain better curl definition and/or regularity, and to improve curl retention. Increasingly, consumers also seek hair products that have a natural look and feel, while imparting good styling benefits to hair. Consumers also seek products that offer multiple benefits, for example, combining frizz reduction and style hold with softening (i.e. not “crunchy” feeling), elongation or lengthening effects while still providing good curl definition. In addition, traditional elongation of hair, including relaxing or straightening hair, often involves chemical treatment or high heat that may cause damage to the hair fibers and/or irritate the scalp.

As such, there is a need for new and improved products and methods for treating keratin fibers, in particular human keratin fibers such as curly or wavy hair, which may make it possible to deliver visible and/or lasting curl definition and/or elongation benefits to hair curls, and/or maintain curls while obtaining good curl definition and/or regularity, particularly under humid conditions, and at the same time impart one or more various additional properties to the hair such as better movement, good volume, frizz control, softness, smoothness, and/or natural shape, and which may be long-lasting, non-irritating, and/or non-toxic.

The present disclosure addresses these concerns and needs, and relates to hair treatment compositions that provide desirable defined, long-lasting curls in hair which can surprisingly be achieved without the need for application of high heat, such as from an iron, blow dryer, or the like, and at the same time, deliver benefits such as improved curl definition, curl regularity, and/or decreased shrinkage, and additional caring and conditioning benefits, such as smoothness, frizz control, volume control, and discipline.

### SUMMARY

The present disclosure relates to compositions for treating, caring for, and styling hair, such as curly hair, that comprise a synergistic combination of various components, including a combination of citric acid and urea or urea derivatives.

It has surprisingly been found that compositions disclosed herein can, in certain embodiments, impart one or more various cosmetic benefits to hair, such as curl definition, curl regularity, curl control/hold, and/or curl elongation (decrease shrinkage), as well as retention of curl/shape, frizz control, volume control, smoothness, curl pick up, discipline, hydration, moisture, shine, conditioning, and other cosmetic benefits to the hair. The compositions may be leave-in compositions or rinse-off compositions, and may be, for example, in the form of a serum, a gel, a gel cream, a cream, a lotion, or the like. The disclosure also relates to packaging systems comprising the compositions, and methods of using the compositions for styling or shaping keratin fibers.

In various embodiments, compositions according to the disclosure provide one or more benefits to hair such as hair styling, and comprise (a) a combination of (i) citric acid and (ii) at least one urea compound chosen from urea and/or derivatives thereof; (b) at least one additional component for styling and/or treating the hair; and (c) at least one solvent. In certain embodiments, components for styling and/or treating the hair may be chosen from silicone compounds, fatty compounds other than silicone compounds (including vegetable oils), cationic surfactants, or mixtures thereof. The compositions may optionally include one or more other components useful in compositions for styling and/or treating the hair, such as, for example, polyols, thickening agents, pH adjustors, preservatives, fragrances, or mixtures thereof. In certain embodiments, the compositions provide both styling and treatment, e.g., conditioning, benefits to hair.

In some embodiments, compositions according to the disclosure comprise (a) a combination of (i) citric acid and (ii) at least one urea compound chosen from urea and/or derivatives thereof; (b) at least one fatty compound; and (c) at least one solvent. The fatty compounds may, for example, be chosen from vegetable oils, fatty alcohols, fatty esters, and other non-silicone fatty compounds, and the compositions may optionally further comprise one or more silicone compounds. The compositions may optionally further comprise additional components, such as polyols, cationic surfactants, thickening agents, emulsifiers, vitamins, and/or hydrating agents. In various embodiments, the pH of the composition is less than about 7.

In other embodiments, compositions according to the disclosure comprise (a) a combination of (i) citric acid and (ii) at least one urea compound chosen from urea and/or derivatives thereof; (b) at least one silicone compound; (c) at least one fatty compound other than silicone compounds and vegetable oils; and (d) at least one solvent. The compositions may optionally further comprise additional components, for example at least one vegetable oil, at least one polyol, at least one cationic surfactant, and/or at least one thickening agent. In various embodiments, the pH of the composition is less than about 7, such as from about 2 to about 6 or from about 3 to about 5.

In various embodiments, the compositions comprise amounts of citric acid and urea compounds in order to provide a mole ratio of citric acid to urea compounds ranging from about 0.1 to about 5, such as from about 0.3 to about 2.5, from about 0.3 to about 2.0, from about 0.4 to about 1.5, or from about 0.5 to about 1.4, or is about 0.45, about 0.6, about 0.7, about 0.75, about 0.8, or about 0.9.

In various embodiments, the compositions comprise amounts of citric acid and urea compounds in order to provide a weight ratio of citric acid to urea compounds ranging from about 1:10 to about 10:1, for example from about 1:8 to about 8:1, from about 1:5 to about 5:1, from about 1:2 to about 2:1, or from about 1:0.95 to about 1:0.05, such as from about 1:0.9 to about 1:0.1, from about 1:0.85 to about 1:0.3, from about 1:0.85 to about 1:0.5, or from about 1:0.8 to about 1:0.6, or is about 1:0.8, about 1:0.75, about 1:0.7, about 1:0.65, or about 1:0.6. In some embodiments, the weight ratio of citric acid to urea compounds is less than about 1, such as less than about 0.75 or less than about 0.5. In further embodiments, the weight ratio of citric acid to urea compounds is greater than about 1, for example ranging from about 1 to about 5. In still further embodiments, the weight ratio of citric acid to urea compounds ranges from about 0.1 to about 5, such as from about 0.2 to about 4.5, or from about 0.3 to about 4.2.

In further embodiments, the total amount of the (a) combination of citric acid and urea compounds is present in the composition in an amount ranging from about 0.5% to about 50%, such as from about 1% to about 20%, from about 1% to about 15%, or from about 1.5% to about 10%, or is about 2%, about 3%, about 3.3%, about 3.5%, about 4%, or about 5% by weight, relative to the total weight of the composition. In certain embodiments, citric acid is present in an amount ranging from about 1% to about 40% by weight, such as from about 1.1% to about 25% or from about 1.1% to about 20%, or is about 1.2%, about 1.4%, about 1.6%, about 1.8%, about 2%, or about 2.5%, relative to the total weight of the composition.

In various embodiments, the at least one urea compound is chosen from urea, dimethyl urea, hydroxylethyl urea, or mixtures thereof. In some embodiments, the at least one urea compound comprises, consists essentially of, or consists of hydroxyethyl urea. In other embodiments, the at least one urea compound comprises, consists essentially of, or consists of dimethyl urea.

In various embodiments, the (a) combination of citric acid and at least one urea compound, for example hydroxylethyl urea, is present in the composition in an amount ranging from about 1.5% to about 5%, and the weight ratio of citric acid to urea compounds, e.g. hydroxylethyl urea, ranges from about 1:5 to about 5:1, such as from about 1:0.85 to about 1:0.5, such as from about 1:0.8 to about 1:0.6, or is about 1:0.8, about 1:0.75, or about 1:0.7.

In various embodiments, the compositions comprise at least one silicone compound, which may, for example, be chosen from dimethicone, dimethicone copolymers, amino functional silicones, or mixtures thereof. The total amount of silicone compounds may vary, but in certain embodiments ranges from about 0.1% to about 10% by weight, such as from about 0.1% to about 5% by weight, relative to the total weight of the composition.

In yet further embodiments, the compositions comprise at least one fatty compound other than silicone compounds, which may, for example, be chosen from fatty alcohols, fatty acids, fatty esters, fatty ethers, derivatives thereof, or mixtures thereof. Exemplary fatty alcohols may include decyl alcohol, undecyl alcohol, dodecyl alcohol, myristyl alcohol, lauryl alcohol, cetyl alcohol, stearyl alcohol, cetearyl alcohol, isostearyl alcohol, isocetyl alcohol, behenyl alcohol, linalool, oleyl alcohol, isotridecyl alcohol, cis-4-t-butylcyclohexanol, myricyl alcohol, or mixtures thereof; exemplary fatty acids may include myristic acid, lauric acid, palmitic acid, stearic acid, behenic acid, arichidonic acid, oleic acid, isostearic acid, sebacic acid, or mixtures thereof; exemplary fatty esters may include cetyl esters, purcellin oil (cetearyl octanoate), isopropyl myristate, isopropyl palmitate, C12-C15 alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, diisostearyl malate, neopentyl glycol dioctanoate, dibutyl sebacate, di-C12-13 alkyl malate, dicetearyl dimer dilinoleate, dicetyl adipate, diisocetyl adipate, diisononyl adipate, diisostearyl dimer dilinoleate, diisostearyl fumarate, or mixtures thereof; exemplary fatty ethers may include olyoxyethylene cetyl/stearyl ether, polyoxyethylene cholesterol ether, polyoxyethylene laurate or dilaurate, polyoxyethylene stearate or distearate, polyoxyethylene lauryl or stearyl ether, dicaprylyl ether, dicetyl ether distearyl ether, dodecyl ether, dilauryl ether, dimyristyl ether, diisononyl ether, or mixtures thereof. In various embodiments, fatty compounds other than silicone compounds may range from about 0.01% to about 30%, such as from about 0.5% to about 30%, from about 1% to about 30%, or from about 5% to about 30% by weight, relative to the total weight of the composition.

The compositions typically include at least one solvent. In various embodiments, the composition comprises water, which may be present in an amount of at least 20% by weight, for example ranging from about 20% to about 90%, from about 30% to about 90%, from about 40% to about 90%, or from about 45% to about 85% by weight, relative to the total weight of the composition. In certain embodiments, the compositions comprise at least one non-aqueous solvent.

In various embodiments, the composition may further optionally comprise at least one vegetable oil, for example may comprise a total amount of vegetable oils ranging from about 0.5% to about 15% by weight, relative to the total weight of the composition. For example, the at least one vegetable oil may be chosen from coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, walnut oil, or mixtures thereof. In one embodiment, the composition comprises coconut and/or avocado oil, which may, in certain embodiments, be present in an amount ranging from about 0.5% to about 15% by weight, relative to the total weight of the composition.

In various embodiments, the composition may further optionally comprise at least one polyol. For example, in various embodiments, the compositions comprise at least one polyol chosen from ethylene glycol, propylene glycol, butylene glycol, hexylene glycol, pentylene glycol, diethylene glycol, dipropylene glycol, 1,3 propanediol, glycerin, polyethylene glycols, or mixtures thereof; wherein the total amount of polyols is at least 0.5%, such as from about 1% to about 10% by weight; relative to the total weight of the composition.

In various embodiments, the composition may further optionally comprise at least one cationic surfactant. For example, the compositions may comprise at least one cationic surfactant chosen from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyldiethylamine, arachidamidoethyldimethylamine, or mixtures thereof; wherein the total amount of cationic surfactants ranges from about 0.1% to about 10% by weight, relative to the total weight of the composition.

In various embodiments, the composition may further optionally comprise at least one thickening agent. For example, the compositions may comprise at least one thickening agent chosen from polysaccharides, which may, for example, be chosen from celluloses, starches, gums, or mixtures thereof, such as chosen from hydroxypropyl guar, xanthan gum, hydroxyethylcelluloses, or mixtures thereof. In various embodiments, the total amount of thickening agents may range from about 0.05% to about 5%, such as from about 0.1% to about 5%, from about 0.1% to about 3% by weight, relative to the total weight of the composition.

In various embodiments, the pH of the composition is less than about 10, such as less than about 7, for example ranges from about 1.5 to about 6.8, from about 2.5 to about 6, from about 3 to about 6.5, from about 3.5 to about 6.5, from about 3.5 to about 6, from about 3.5 to about 5.5, from about 3.5 to about 5, from about 3.5 to about 4.5, from about 3.5 to about 4, or from about 3.5 to about 4. Optionally, the compositions may comprise at least one pH adjustor.

In various embodiments, the compositions may be leave-in compositions for styling hair, for example by improving curl definition and/or curl regularity, and/or by elongating curls of curly hair. The compositions may additionally, in some embodiments, provide at least one hair treatment benefit, for example imparting smoothness, softness, and/or sheen to the hair.

The disclosure further relates to compositions for improving curl definition and/or curl regularity of curly hair comprising (a) from about 1.0% to about 15%, such as from about 1.5% to about 10%, of a combination of (i) citric acid and (ii) at least one urea compound chosen from urea and/or derivatives thereof, wherein a weight ratio of citric acid to urea compounds is at least than 1; (b) from about 0.5% to about 15%, such as from about 0.5% to about 10% of at least one vegetable oil; (c) optionally, from about 0.1% to about 10%, such as from about 0.5% to about 5%, of at least one silicone compound; (d) from about 1% to about 30%, such as about 10% to about 30% of at least one fatty compound chosen from fatty alcohols, fatty acids, fatty esters, fatty ethers, derivatives thereof, or mixtures thereof; and (e) at least 0.5%, such as about 1% to about 20% of at least one polyol; and (f) from about 40% to about 85% of water, wherein all percentages are by weight and are based on the total weight of the composition; wherein the pH of the composition is less than about 10, for example ranges from about 2 to about 6.5, such as from about 2.5 to about 6, from 3 to about 5, or from about 3 to about 4.

The disclosure further relates to a composition for improving elongation and curl definition of curly hair comprising (a) from about 1.0% to about 15%, such as from about 1.5% to about 10%, of a combination of (i) citric acid and (ii) at least one urea compound chosen from urea and/or derivatives thereof, wherein a mole ratio of citric acid to urea compounds ranges from about 0.4:1 to about 3:1; (b) from about 0.5% to about 15%, such as from about 0.5% to about 10%, of at least one vegetable oil; (c) optionally, from about 0.1% to about 10%, such as from about 0.5% to about 5%, of at least one silicone compound; (d) from about 1% to about 30%, such as about 1% to about 15%, of at least one fatty compound chosen from fatty alcohols, fatty acids, fatty esters, fatty ethers, derivatives thereof, or mixtures thereof; (e) from about 0.1% to about 10% of at least one cationic surfactant; and (f) from about 50% to about 85%, such as from about 60% to about 85%, from about 70% to about 85%, of water; wherein all percentages are by weight and are based on the total weight of the composition; wherein the pH of the composition is less than about 10, for example ranges from about 2 to about 5, such as from about 3.5 to about 4.5, from about 3.5 to about 4.

The disclosure further relates to methods of using the compositions described herein to impart one or more various styling or care/treatment benefits, such as curl definition, curl regularity, curl control/hold, curl elongation, retention of curl/shape, frizz control, volume control, smoothness, curl pick up, discipline, hydration, moisture, shine, and/or conditioning, to hair, by applying a composition according to the disclosure to hair.

For example, the disclosure relates to methods for styling hair, in particular curly hair, wherein the methods may improve curl definition, improve curl regularity, and/or improve curl elongation, and may optionally impart one or more treatment effects, e.g. conditioning. The methods generally comprise applying to the hair a styling composition according to the disclosure to the hair. The composition may be allowed to remain on the hair for any desired period of time for improving curl definition and/or curl regularity, and/or elongating curls of curly hair, and/or for imparting treatment effects to the hair.

The disclosure further relates to kits for treating, caring for, and/or styling curly hair comprising at least one container containing a first composition which is a composition according to the disclosure; and optionally, at least one additional container containing a second composition, which is a composition according to the disclosure or a composition other than a composition according to the disclosure.

The disclosure further relates to a method of producing a composition for treating, caring for, and/or styling curly hair, the method comprising mixing citric acid and at least one urea compound chosen from urea and/or derivatives thereof to obtain a mixture thereof, wherein a mole ratio of citric acid to urea compounds in the mixture ranges from about 0.4:1 to about 3; and adding about 1% to about 20%, such as about 1% to about 15%, or from about 1.5% to about 10%, of the mixture to a base composition, which may, for example, comprise at least one component for styling and/or treating hair, such as, for example: at least one silicone compound; at least one fatty compound other than silicone compounds; at least one aqueous and/or non-aqueous solvent; at least one vegetable oil; at least one polyol; at least one cationic surfactant; at least one thickening agent; or mixtures thereof.

### BRIEF DESCRIPTION OF FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a set of images showing a hair swatch treated with a composition according to the disclosure in comparison with hair swatches treated with comparative compositions and a control.

FIG. 2 is a set of images showing a hair swatch treated with a composition according to the disclosure in comparison with hair swatches treated with comparative compositions.

It is to be understood that the foregoing and following descriptions are exemplary and explanatory only and are not intended to be restrictive of any subject matter claimed.

### DETAILED DESCRIPTION

The disclosure relates to compositions for treating, caring for, and/or styling hair, in particular, for improving curl definition, curl regularity, and elongation of curly hair, as well as kits comprising the compositions, methods of using the compositions, and methods of producing the compositions.

A unique feature of the compositions and methods according to the disclosure is that a synergistic combination of citric acid and at least one urea compound chosen from urea and/or derivatives thereof in specific weight ratios or mole ratios is integrated into compositions for treating or styling curly hair. The integration of the combination of citric acid and urea compounds with a specific weight ratio in compositions as disclosed herein surprisingly provides improved curl definition, curl regularity, elongation (decreased shrinkage), smoothness, and/or frizz control, as well as other beneficial sensorial attributes, to the hair.

I. Compositions

In various exemplary and non-limiting embodiments, compositions according to the disclosure provide hair styling benefits and comprise (a) a combination of (i) citric acid and (ii) at least one urea compound chosen from urea and/or derivatives thereof; wherein a mole ratio of citric acid to urea compound(s) ranges from about 0.1 to about 5, such as from about 0.4 to about 3; (b) at least one additional component for styling and/or treating the hair; and (c) at least one solvent. In some embodiments, the weight ratio of citric acid to urea compound(s) may be at least about 0.1, such as at least about 0.2, at least about 0.3, at least about 0.4, at least about 0.5, or at least about 1, for example may be greater than about 1, such as greater than 1.

In other exemplary and non-limiting embodiments, compositions according to the disclosure provide hair styling benefits and comprise (a) a combination comprising citric acid and at least one urea compound chosen from urea and/or a urea derivative, wherein the mole ratio of citric acid to urea compound(s) ranges from about 0.4 to about 3; (b) at least one silicone compound; and (c) at least one fatty compound other than silicone compounds; and (d) at least one solvent. In some embodiments, the weight ratio of citric acid to urea compound(s) may be at least about 1, for example may be greater than about 1, such as greater than 1. The compositions may further optionally comprise (e) at least one vegetable oil; (f) at least one polyol; (g) at least one cationic surfactant; and/or (h) at least one thickening agent; as well as certain other additional components.

The compositions may, in some embodiments, be free or essentially free of silicone compounds and/or sulfate-based anionic surfactants.

Compositions according to the disclosure may improve hair curl definition, curl regularity, and/or curl hold, may provide hair curl elongation, and/or may provide additional benefits such as softness, smoothness, and/or frizz control to the hair. The compositions described herein are particularly suited to multicultural hair types of varying curliness and hair types. The degree of curliness or curl type of the hair may vary and is not limited.

### Synergistic Combination of Citric Acid and Urea Compounds

Compositions according to the disclosure comprise a surprisingly synergistic combination of citric acid and at least one urea compound chosen from urea and/or derivatives thereof.

As used herein, a “urea compound” refers to urea or a urea derivative chosen from compounds of the following formula (I):

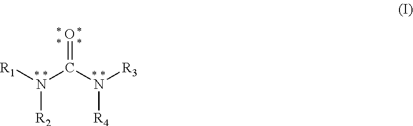

wherein R₁, R₂, R₃, and R₄ are independently chosen from hydrogen, C₄ to C₁₀ substituted or unsubstituted aryl groups, C₂ to C₁₀ substituted or unsubstituted heterocycle groups, C₁ to C₁₀ substituted or unsubstituted alkyl groups, and C₃ to C₁₀ substituted or unsubstituted cycloalkyl groups.

Non-limiting examples of suitable urea derivatives include imidazolidinyl urea, diazolidinyl urea, m-dimethylaminophenyl urea, dimethyl urea, a hydroxyethyl urea, urea, urea derivatives, imidazolidinyl urea, diazolidinyl urea, m-dimethylaminophenyl urea, dimethyl urea, a hydroxyethyl urea, N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(tris-hydroxymethylmethyl)urea; N-ethyl-N′-(2-hydroxyethyl)urea; N,N-bis(2-hydroxyethyl)urea; N, N′-bis(2-hydroxyethyl)urea; N,N-bis(2-hydroxypropyl)urea; N, N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′, and N′-bis(2-hydroxypropyl)-urea.

In some embodiments, the at least one urea compound in the combination with citric acid is chosen from urea, dimethyl urea, hydroxyethyl urea, or mixtures thereof. In certain preferred embodiments, the at least one urea compound comprises, consists essentially of, or consists of hydroxyethyl urea.

As used herein, the term “citric acid” includes salts of citric acid, whether or not expressly stated. In various exemplary embodiments, the total amount of citric acid, salts thereof, or mixtures thereof (referred to herein collectively as “citric acid”) that may be included in the compositions disclosed herein may range from about 0.1% to about 50%, including all subranges therebetween, such as from about 0.1% to about 40%, from about 0.1% to about 30%, from about 0.1% to about 20%, from about 0.1% to about 15%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.1% to about 0.75%, from about 0.1% to about 0.5%, from about 0.15% to about 10%, from about 0.15% to about 9%, from about 0.15% to about 8%, from about 0.15% to about 7%, from about 0.15% to about 6%, from about 0.15% to about 5%, from about 0.15% to about 4%, from about 0.15% to about 3%, from about 0.15% to about 2%, from about 0.15% to about 1%, from about 0.15% to about 0.75%, from about 0.15% to about 0.5%, from about 0.2% to about 10%, from about 0.2% to about 9%, from about 0.2% to about 8%, from about 0.2% to about 7%, from about 0.2% to about 6%, from about 0.2% to about 5%, from about 0.2% to about 4%, from about 0.2% to about 3%, from about 0.2% to about 2%, from about 0.2% to about 1.5%, from about 0.2% to about 1%, from about 0.2% to about 0.75%, from about 0.2% to about 0.5%, from about 0.3% to about 5%, from about 0.3% to about 4%, from about 0.3% to about 3%, from about 0.3% to about 2.5%, from about 0.3% to about 2%, from about 0.3% to about 1.5%, from about 0.3% to about 1%, from about 0.3% to about 0.75%, from about 0.3% to about 0.5%, from about 0.5% to about 40%, about 0.5% to about 35%, about 0.5% to about 30%, about 0.5% to about 35%, about 0.5% to about 30%, about 0.5% to about 25%, about 0.5% to about 20%, about 0.5% to about 15%, about 0.5% to about 12%, about 0.5% to about 10%, about 0.5% to about 8%, about 0.5% to about 6%, about 0.5% to about 5%, about 0.5% to about 4%, about 0.5% to about 3%, about 0.5% to about 2.5%, about 0.5% to about 2%, about 0.5% to about 1.5%, about 0.5% to about 1%, about 0.8% to about 40%, about 0.8% to about 35%, about 0.8% to about 30%, about 0.8% to about 35%, about 0.8% to about 30%, about 0.8% to about 25%, about 0.8% to about 20%, about 0.8% to about 15%, about 0.8% to about 12%, about 0.8% to about 10%, about 0.8% to about 8%, about 0.8% to about 6%, about 0.8% to about 5%, about 0.8% to about 4%, about 0.8% to about 3%, about 0.8% to about 2%, about 0.5% to about 40%, about 0.5% to about 35%, about 0.5% to about 30%, about 0.5% to about 20%, about 0.5% to about 10%, about 0.5% to about 8%, about 1% to about 25%, about 1% to about 20%, about 1% to about 15%, about 1% to about 12%, about 1% to about 15%, about 1% to about 10%, about 1% to about 8%, about 1% to about 6%, about 1% to about 5%, about 1% to about 4%, about 1% to about 3%, about 1% to about 2%, about 1.2% to about 15%, about 1.2% to about 12%, about 1.2% to about 10%, about 1.2% to about 8%, about 1.2% to about 6%, about 1.2% to about 5%, about 1.2% to about 4%, about 1.2% to about 3%, or about 1.2% to about 2% based on the total weight of the composition.

In some embodiments, the total amount of citric acid is less than about 1%, for example less than about 0.8%, less than about 0.5%, or less than about 0.3%, for example ranges from about 0.1% to about 1%, from about 0.25% to about 1%, from about 0.5% to about 0.9%, or from about 0.6% to about 0.8% by weight, relative to the total weight of the composition. In other embodiments, the total amount of citric acid is less than about 3%, for example less than about 2.5%, less than about 2%, or less than about 1.5%, for example ranges from about 0.1% to about 3%, from about 0.2% to about 2.8%, or from about 0.25% to about 2.5% by weight, relative to the total weight of the composition.

In other embodiments, the total amount of citric acid is greater than about 1%, for example ranges from about 1% to about 10%, and such as ranges from about 1% to about 5%, from about 1% to about 4%, or from about 1% to about 3.5% by weight, or ranges from greater than 1% to about 10%, such as from greater than 1% to about 5%, from greater than 1% to about 4%, or from greater than 1% to about 3.5% by weight, relative to the total weight of the composition. In further embodiments, the total amount of citric acid present in the composition is about 1%, about 1.2%, about 1.4%, about 1.6%, about 1.8%, about 2%, or about 2.5% by weight, relative to the total weight of the composition.

In various exemplary embodiments, the total amount of urea, urea derivatives, and mixtures thereof (referred to herein collectively as “urea compounds”) that may be included in the compositions disclosed herein may range from about 0.01% to about 25%, including all subranges therebetween, such as from about 0.1% to about 20%, from about 0.1% to about 18%, from about 0.1% to about 15%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, about 0.2% to about 20%, from about 0.2% to about 18%, from about 0.2% to about 15%, from about 0.05% to about 10%, from about 0.05% to about 9%, from about 0.05% to about 8%, from about 0.05% to about 7%, from about 0.05% to about 6%, from about 0.05% to about 5%, from about 0.05% to about 4%, from about 0.05% to about 3%, from about 0.05% to about 2%, from about 0.1% to about 15%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.2% to about 15%, from about 0.2% to about 10%, from about 0.2% to about 9%, from about 0.2% to about 8%, from about 0.2% to about 7%, from about 0.2% to about 6%, from about 0.2% to about 5%, from about 0.2% to about 4%, from about 0.2% to about 3%, from about 0.2% to about 2%, from about 0.4% to about 20%, from about 0.4% to about 18%, from about 0.4% to about 15%, from about 0.4% to about 10%, from about 0.4% to about 9%, from about 0.4% to about 8%, from about 0.4% to about 7%, from about 0.4% to about 6%, from about 0.4% to about 5%, from about 0.4% to about 4%, from about 0.4% to about 3%, from about 0.4% to about 2%, or from about 0.4% to about 1.5% by weight, or is about 0.5%, about 0.8%, about 1%, about 1.2%, about 1.4%, about 1.6%, about 1.8%, or about 2% by weight, relative to the total weight of the composition.

In some embodiments, the total amount of urea compounds is less than about 5%, such as less than about 4%, or less than about 3% by weight, relative to the total weight of the composition. For example, in some embodiments, the total amount of urea compounds ranges from about 0.1% to about 5%, such as from about 0.2% to about 4%, from about 0.3% to about 3%, or from about 0.5% to about 2.5% by weight, relative to the total weight of the composition.

In some embodiments, the compositions comprise a total amount of citric acid ranging from about 1% to about 10%, such as from about 1% to about 7%, from about 1% to about 5%, or from about 1% to about 3.5% by weight, and a total amount of urea compounds ranging from about 0.05% to about 5%, such as from about 0.1% to about 4%, from about 0.2% to about 3%, or from about 0.3% to about 2% by weight, relative to the total weight of the composition. In other embodiments, the compositions comprise a total amount of citric acid ranging from about 0.1% to about 5%, such as from about 0.1% to about 3%, or about 0.1% to about 1%, and a total amount of urea compounds ranging from about 0.1% to about 5%, such as from about 0.2% to about 4%, from about 0.3% to about 3%, or from about 0.4% to about 2% by weight, relative to the total weight of the composition.

In various embodiments, the total amounts of citric acid and urea compounds may be chosen so that the weight ratio of the citric acid to the urea compounds allows the combination thereof to provide synergistic optimal hair styling and/or care benefits. In various embodiments of the disclosure, citric acid is typically included in an amount of at least the same or greater than the amount of urea compounds. For instance, in certain embodiments, the weight ratio of the total amount of citric acid to urea compounds is less than about 1, such as less than about 0.9, less than about 0.8, less than about 0.7, less than about 0.6, less than about 0.5, less than about 0.4, or less than about 0.35. For example, the weight ratio of the total amount of citric acid to urea compounds may be about 0.1, about 0.2, about 0.3, about 0.4, about 0.5, about 0.7, about 0.7, about 0.8, about 0.9, or about 1, or may be within any range using the foregoing as upper and lower limits.

In other embodiments, the weight ratio of the total amount of citric acid to the amount of urea compounds may range from about 1:1 to about 1:0.05, including all subranges therebetween, such as from about 1:0.95 to about 1:0.1, from about 1:0.95 to about 1:0.2, from about 1:0.95 to about 1:0.4, from about 1:0.95 to about 1:0.6, from about 1:0.95 to about 1:0.8, from about 1:0.9 to about 1:0.1, from about 1:0.9 to about 1:0.2, from about 1:0.9 to about 1:0.4, from about 1:0.9 to about 1:0.6, from about 1:0.85 to about 1:0.05, from about 1:0.85 to about 1:0.1, from about 1:0.85 to about 1:0.3, from about 1:0.85 to about 1:0.4, from about 1:0.85 to about 1:0.6, from about 1:0.8 to about 1:0.05, from about 1:0.8 to about 1:0.1, from about 1:0.8 to about 1:0.2, from about 1:0.8 to about 1:0.3, from about 1:0.8 to about 1:0.4, from about 1:0.8 to about 1:0.5, or from about 1:0.8 to about 1:0.6. In some embodiments, the weight ratio of the total amount of citric acid to the urea compounds is about 1:0.25, about 1:0.3, about 1:0.35, about 1:0.4, about 1:0.45, about 1:0.5, about 1:0.55, about 1:0.6, about 1:0.65, about 1:0.7, about 1:0.75, or about 1:0.8.

In some embodiments, the composition may be formulated to have a weight ratio of the total amounts of citric acid to urea compounds ranging from about 10:1 to about 1:10, such as from about 5:1 to about 1:5, from about 3:1 to about 1:3, or from about 1.5:1 to about 1:1.5. In some instances, the cosmetic composition may be formulated to have a weight ratio of the total amounts of citric acid to urea compounds of about 10:1 to about 0.5:10, about 9:1 to about 0.5:10, about 8:1 to about 0.5:10, about 7:1 to about 0.5:10, about 6:1 to about 0.5:10, about 5:1 to about 0.5:10, about 4:1 to about 0.5:10, about 3:1 to about 0.5:10; 10:1 to about 1:10, about 9:1 to about 1:10, about 8:1 to about 1:10, about 7:1 to about 1:10, about 6:1 to about 1:10, about 5:1 to about 1:10, about 4:1 to about 1:10, about 3:1 to about 1:10; 10:1 to about 2:10, about 9:1 to about 2:10, about 8:1 to about 2:10, about 7:1 to about 2:10, about 6:1 to about 2:10, about 5:1 to about 2:10, about 4:1 to about 2:10, about 3:1 to about 2:10; about 10:1 to about 2:8, about 9:1 to about 2:8, about 8:1 to about 2:8, about 7:1 to about 2:8, about 6:1 to about 2:8, about 5:1 to about 2:8, about 4:1 to about 2:8, about 3:1 to about 2:8; about 10:1 to about 2:6, about 9:1 to about 2:6, about 8:1 to about 2:6, about 7:1 to about 2:6, about 6:1 to about 2:6, about 5:1 to about 2:6, about 4:1 to about 2:6, about 3:1 to about 2:6; about 3:1 to about 1:10, about 3:1 to about 1:9, about 3:1 to about 1:8, about 3:1 to about 1:7, about 3:1 to about 1:6, about 3:1 to about 2:10, about 3:1 to about 2:9, about 3:1 to about 2:8, about 3:1 to about 2:7, about 3:1 to about 2:6, about 3:1 to about 2:5, about 3:1 to about 2:4, or about 3:1 to about 2:3, about 2:1 to about 2:10, about 1:1 to about 2:10, about 3:1 to about 2:9, about 3:1 to about 2:8, about 3:1 to about 2:7, about 3:1 to about 2:6, about 3:1 to about 2:5, about 3:1 to about 2:4, or about 3:1 to about 2:3 including ranges and sub-ranges there between (e.g., about 3:1 to about 2:5, about 2:1 to about 2:5, about 1:1 to about 2:5, about 1:1 to about 2:4, etc.).

The composition may be formulated to have a weight ratio of the total amounts of citric acid to urea compounds that may range from about 10:1 to about 0.5:10. In some instances, the composition may be formulated to have a weight ratio of the total amounts of citric acid to urea compounds ranging from about 10:1 to about 0.5:10, about 9:1 to about 0.5:10, about 8:1 to about 0.5:10, about 7:1 to about 0.5:10, about 6:1 to about 0.5:10, about 5:1 to about 0.5:10, about 4:1 to about 0.5:10, about 3:1 to about 0.5:10; 10:1 to about 1:10, about 9:1 to about 1:10, about 8:1 to about 1:10, about 7:1 to about 1:10, about 6:1 to about 1:10, about 5:1 to about 1:10, about 4:1 to about 1:10, about 3:1 to about 1:10; about 10:1 to about 2:10, about 9:1 to about 2:10, about 8:1 to about 2:10, about 7:1 to about 2:10, about 6:1 to about 2:10, about 5:1 to about 2:10, about 4:1 to about 2:10, about 3:1 to about 2:10; about 10:1 to about 2:8, about 9:1 to about 2:8, about 8:1 to about 2:8, about 7:1 to about 2:8, about 6:1 to about 2:8, about 5:1 to about 2:8, about 4:1 to about 2:8, about 3:1 to about 2:8; about 10:1 to about 2:6, about 9:1 to about 2:6, about 8:1 to about 2:6, about 7:1 to about 2:6, about 6:1 to about 2:6, about 5:1 to about 2:6, about 4:1 to about 2:6, about 3:2 to about 2:6; about 2:1 to about 2:10, about 1:1 to about 2:10, about 3:2 to about 2:9, about 3:2 to about 2:8, about 3:2 to about 2:7, about 3:2 to about 2:6, about 3:2 to about 2:5, about 3:2 to about 2:4, about 3:2 to about 2:3, about 1:1 to about 1:4, about 1:1 to about 1:3, about 1:1 to about 1:2, or about 1:1.3 to about 1:1.6, including ranges and sub-ranges therebetween (e.g., about 3:2 to about 2:5, about 2:1 to about 2:5, about 1:1 to about 2:5, about 1:1 to about 2:4, etc.).

In some other embodiments, the mole ratio of citric acid to urea compounds may range from about 0.1 to about 5, such as from about 0.3 to about 2.5, such as from about 0.3 to about 2.0, from about 0.4 to about 1.5, or from about 0.5 to about 1.4, or may be about 0.45, about 0.6, about 0.7, about 0.75, about 0.8, or about 0.9.

In some embodiments, the combination of citric acid and the at least one urea compound may be in the form of a pre-phase mixture or pre-mix. For example, the combination of citric acid and at least one urea compound may be produced by combining and mixing particular amounts of citric acid and urea compounds with specific mole and/or weight ratios as described herein to form a mixture. The pre-phase mixture can subsequently be added to a base composition comprising other cosmetic ingredients to produce the compositions according to the disclosure for styling and/or treating hair, such as curly hair. In some embodiments, however, the citric acid and at least one urea compound are not prepared as a pre-phase mixture.

In various exemplary embodiments, the total amount of the pre-phase mixture of citric acid and at least one urea compound may be included in a composition according to the disclosure in an amount ranging from about 0.5% to about 50%, including all ranges and subranges therebetween, such as from about 0.5% to about 40%, from about 0.5% to about 30%, from about 0.5% to about 20%, from about 0.5% to about 15%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3.5%, from about 1% to about 50%, from about 1% to about 40%, from about 1% to about 30%, from about 1% to about 20%, from about 1% to about 15%, from about 1% to about 10%, from about 1% to about 9%, from about 1% to about 8%, from about 1% to about 7%, from about 1% to about 6%, from about 1% to about 5%, from about 1% to about 4%, from about 1% to about 3.5%, from about 1.5% to about 50%, from about 1.5% to about 40%, from about 1.5% to about 30%, from about 1.5% to about 20%, from about 1.5% to about 15%, from about 1.5% to about 10%, about 1.5% to about 9%, about 1.5% to about 8%, about 1.5% to about 7%, about 1.5% to about 6%, from about 1.5% to about 5%, from about 1.5% to about 4%, from about 1.5% to about 3.5%, from about 2% to about 50%, from about 2% to about 40%, from about 2% to about 30%, from about 2% to about 20%, from about 2% to about 15%, from about 2% to about 10%, from about 2% to about 9%, from about 2% to about 8%, from about 2% to about 7%, from about 2% to about 6%, from about 2% to about 5%, from about 2% to about 4%, from about 2% to about 3.5%, from about 2.5% to about 50%, from about 2.5% to about 40%, from about 2.5% to about 30%, from about 2.5% to about 20%, from about 2.5% to about 15%, from about 2.5% to about 10%, about 2.5% to about 9%, about 2.5% to about 8%, about 2.5% to about 7%, about 2.5% to about 6%, from about 2.5% to about 5%, from about 2.5% to about 4%, from about 2.5% to about 3.5%, from about 3% to about 50%, from about 3% to about 40%, from about 3% to about 30%, from about 3% to about 20%, from about 3% to about 15%, from about 3% to about 10%, from about 3% to about 9%, from about 3% to about 8%, from about 3% to about 7%, from about 3% to about 6%, from about 3% to about 5%, from about 3% to about 4%, from about 3% to about 3.5%, from about 3.5% to about 50%, from about 3.5% to about 40%, from about 3.5% to about 30%, from about 3.5% to about 20%, from about 3.5% to about 15%, from about 3.5% to about 10%, about 3.5% to about 9%, about 3.5% to about 8%, about 3.5% to about 7%, about 3.5% to about 6%, from about 3.5% to about 5%, from about 4% to about 50%, from about 4% to about 40%, from about 4% to about 30%, from about 4% to about 20%, from about 4% to about 15%, from about 4% to about 10%, from about 4% to about 9%, from about 4% to about 8%, from about 4% to about 7%, from about 4% to about 6%, or from about 4% to about 5%, or is about 1%, about 2%, about 3%, about 4%, or about 5% by weight, relative to the total weight of the composition. In some embodiments, the total amount of the pre-phase mixture of citric acid and at least one urea compound included in a composition disclosed herein is about 3.1%, about 3.2%, about 3.3%, about 3.4%, or about 3.5% by weight, relative to the total weight of the composition, including all ranges and subranges thereof, and in other embodiments the total amount of the combination of citric acid and at least one urea compound included in a composition disclosed herein is about 4.8%, about 4.9%, about 5.0%, about 5.1%, or about 5.2% by weight, relative to the total weight of the composition, including all ranges and subranges thereof.

In some embodiments, an exemplary composition disclosed here comprises any of the above ranges, such as from about 0.5% to about 10%, from about 1% to about 7%, from about 2% to about 6%, or from about 3% to about 5.5%, for example is about 3.3% or about 5%, of a pre-phase mixture of citric acid and hydroxyethyl urea, where a weight ratio of citric acid to hydroxyethyl urea in the pre-phase mixture ranges from about 1:1 to about 1:0.05, such as about 1:1 to about 1:0.5, or is about 1:0.75, corresponding to a mole ratio of citric acid to hydroxyethyl urea that is about 1:1.4.

### Silicone Compounds

Compositions according to the disclosure may optionally comprise at least one silicone compound. Without intending to be limited by theory, the silicone compounds may provide treatment or care benefits such as flexibility, manageability, discipline, an overall softer end feel, etc., to the hair. Thus, in some embodiments, the compositions comprise at least one silicone compound, particularly compositions for providing treatment or care benefits to the hair. By way of example only, the at least one silicone compound may be chosen from dimethicone, dimethicone copolyols, dimethicone copolymers, amino functional silicones, or mixtures thereof. In some embodiments, however, the compositions are free or substantially free of silicone compounds.

In some embodiments, the at least one silicone compound is chosen from dimethicone copolyols. Dimethicone copolyol is a general term used for a group of polymers made from dimethicone and polyoxyethylene and/or polyoxypropylene. Suitable examples of dimethicone copolyols include Dimethicone PEG-8 Adipate, Dimethicone PEG-8 Benzoate, Dimethicone PEG-7 Phosphate, Dimethicone PEG-8 Phosphate, Dimethicone PEG-10 Phosphate, Dimethicone PEG/PPG-20/23 Benzoate, Dimethicone PEG/PPG-7/4 Phosphate, Dimethicone PEG/PPG-12/4 Phosphate, PEG-3 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-6/11 Dimethicone, PEG/PPG-8/14 Dimethicone, PEG/PPG-14/4 Dimethicone, PEG/PPG-15/15 Dimethicone, PEG/PPG-16/2 Dimethicone, PEG/PPG-17/18 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, and mixtures thereof.

In some embodiments, the at least one silicone compound may be an amino functional silicone. In an embodiment, the at least one silicone compound that can be chosen is amino functional silicone comprising at least one functionalized amodimethicone. The term “amino functional silicone” as used herein can mean any silicone comprising at least one primary, secondary or tertiary amine or a quaternary ammonium group (i.e., a quaternized group).

Non-limiting examples of amino functional silicone that may be used include:

a) polysiloxanes corresponding to formula (A):

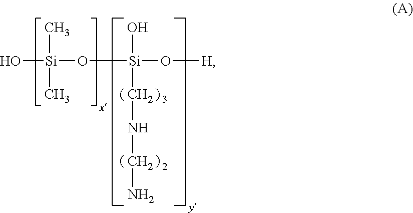

wherein x′ and y′ are integers such that the weight-average molecular weight (Mw) ranges from about 5000 to about 500 000;

b) amino silicones corresponding to formula (B):

R′ₐG₃₋ₐ-Si(OSiG₂)ₙ-(OSiG_{b}R′_{2-b})ₘ—O—SiG₃₋ₐ-R′ₐ (B)

wherein:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10; and
R′, which may be identical or different, denote a monovalent radical having formula —CqH₂qL, in which q is a number ranging from 2 to 8 and L is an optionally quaternized amino group chosen from the following groups:
—NR″-Q-N(R″)₂
—N(R″)₂
—N⁺(R″)₃A⁻
—N⁺H(R″)₂A⁻
—N⁺H₂(R″)A⁻
—N(R″)-Q-N⁺R″H₂A⁻
—NR″-Q-N⁺(R″)₂H A⁻
—NR″-Q-N⁺(R″)₃A⁻,
   in which:
   R″, which may be identical or different, denotes hydrogen, phenyl, benzyl, or a saturated monovalent hydrocarbon-based radical, for example a C₁-C₂₀ alkyl radical;
   Q denotes a linear or branched CᵣH₂ᵣ group, r being an integer ranging from 2 to 6, such as from 2 to 4; and
   A⁻ represents a cosmetically acceptable ion, in particular a halide such as fluoride, chloride, bromide or iodide.

A group of amino silicones corresponding to formula (B) is represented by the silicones called “trimethylsilylamodimethicone” having formula (C):

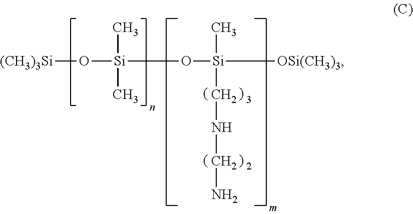

in which n and m have the meanings as in formula B.

Another group of amino silicones corresponding to formula (B) is represented by silicones of formula (D):

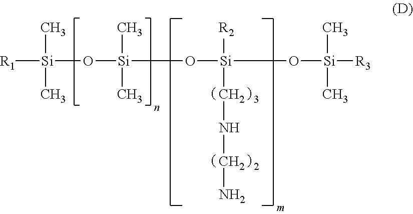

wherein:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and form to denote a number from 1 to 1000 and in particular from 1 to 10, and more particularly from 1 to 5; and
R1, R2, and R3, which may be identical or different, represent a hydroxy or C1-C4 alkoxy radical, where at least one of the radicals R1 to R3 denotes an alkoxy radical.

In one embodiment, the alkoxy radical is a methoxy radical. In further embodiments, the hydroxy/alkoxy mole ratio ranges from 0.2:1 to 0.4:1 or from 0.25:1 to 0.35:1, for example equals 0.3:1. In various embodiments, the weight-average molecular weight (Mw) of the silicone ranges from 2000 to 1,000,000, such as from 3500 to 200,000.

Another group of amino silicones corresponding to formula (B) is represented by silicones of formula (E):

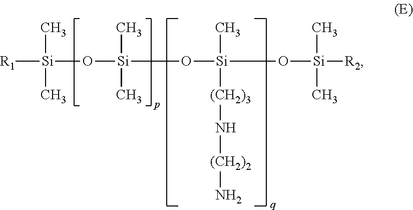

wherein:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5; and
R₁ and R₂, which are different, represent a hydroxy or C₁-C₄ alkoxy radical, where at least one of the radicals R₁ or R₂ denotes an alkoxy radical.

In one embodiment, the alkoxy radical is a methoxy radical. In further embodiments, the hydroxy/alkoxy mole ratio ranges generally from 1:0.8 to 1:1.1 or from 1:0.9 to 1:1, or for example equals 1:0.95. In various embodiments, the weight-average molecular weight (Mw) of the silicone ranges from 2000 to 200,000, such as from 5000 to 100,000 or from 10,000 to 50,000.

Commercial products corresponding to these silicones having structure (D) or (E) may include in their composition one or more other amino silicones whose structure is different than formulae (D) or (E). For example, a product containing amino silicones having structure (D) is sold by Wacker under the name Belsil® ADM 652, and products containing amino silicones having structure (E) include those sold by Wacker under the names Fluid WR 1300® or Finish CT 96 E® or SLM 28020®.

Another group of amino silicones corresponding to formula (B) is represented by the following formula (F), which may be linear:

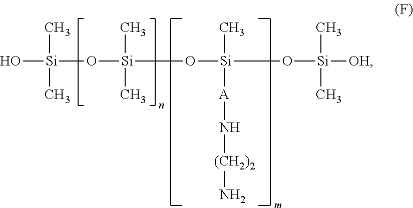

wherein:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10; and
A denotes a linear or branched alkylene radical containing from 4 to 8 carbon atoms, e.g. may have 4 carbon atoms.

The weight-average molecular weight (Mw) of these amino silicones ranges from 2000 to 1,000,000 or from 3500 to 200,000. A preferred silicone of formula (F) is amodimethicone (INCI name) sold under the tradename XIAMETER® MEM-8299 Cationic Emulsion by Dow Corning or sold under the tradename SILSOFT 253, by Momentive Performance Materials.

Another group of amino silicones corresponding to formula (B) is represented by the following formula (G), which is optionally branched:

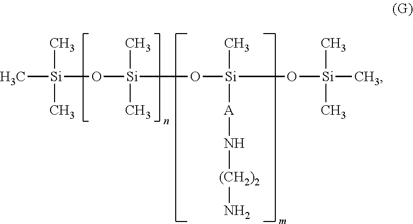

wherein:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10; and
A denotes a linear or branched alkylene radical containing from 4 to 8 carbon atoms, e.g. may have 4 carbon atoms.

The weight-average molecular weight (Mw) of these amino silicones from 500 to 1,000,000 or from 1000 to 200,000. Commercially available silicones having this formula include DC2-8566 Amino Fluid by Dow Corning.

c) amino silicones corresponding to formula (H):

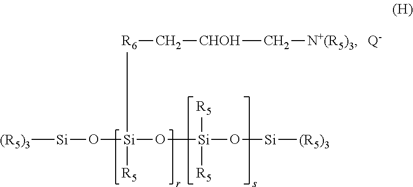

wherein:
R5 represents a monovalent hydrocarbon-based radical containing from 1 to 18 carbon atoms, and in particular a C₁-C₁₈ alkyl or C₂-C₁₈ alkenyl radical, for example methyl;
R6 represents a divalent hydrocarbon-based radical, in particular a C₁-C₁₈ alkylene radical or a divalent C₁-C₁₈, for example C₁-C₈, alkylenoxy radical linked to the Si via an Si—C bond;
Q⁻ is an anion such as a halide ion, in particular chloride, or an organic acid salt (for example acetate);
r represents a mean statistical value from 2 to 20 and in particular from 2 to 8; and
s represents a mean statistical value from 20 to 200 and in particular from 20 to 50.

d) quaternary ammonium silicones having formula (I):

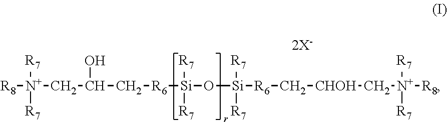

wherein:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an Si—C bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X⁻ is an anion such as a halide ion, in particular chloride, or an organic acid salt (for example acetate); and
r represents a mean statistical value from 2 to 200 and in particular from 5 to 100.
   Examples of such silicones are described, for example, in EP-A 0 530 974.

e) amino silicones having formula (J):

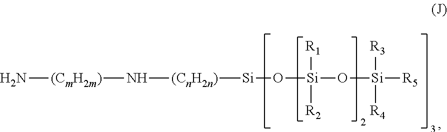

wherein:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5; and
x is chosen such that the amine number is between 0.01 and 1 meq/g.

f) multiblock polyoxyalkylenated amino silicones of type (AB)ₙ, A being a polysiloxane block and B being a polyoxyalkylenated block containing at least one amine group. In various embodiments, such silicones may comprise repeating units having one of the following general formulae:

[—(SiMe₂O)xSiMe₂-R—N(R″)—R′—O(C₂H₄₀)ₐ(C₃H₆O)b-R′—N(H)—R—]

[—(SiMe₂O)xSiMe₂-R—N(R″)—R′—O(C₂H₄O)ₐ(C₃H₆O)b-]

wherein:
a is an integer greater than or equal to 1, such as ranging from 5 to 200, more particularly ranging from 10 to 100;
b is an integer comprised between 0 and 200, such as ranging from 4 to 100, more particularly between from 5 and 30;
x is an integer ranging from 1 to 10,000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; for example R may denote an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical; and
R′, which may be identical or different, represent a divalent linear or branched C₂-C₁₂ hydrocarbon-based radical, optionally including one or more heteroatoms such as oxygen; such as, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks may represent from 50-95 mol % of the total weight of the silicone, more particularly from 70-85 mol %. The amine content may be from 0.02 to 0.5 meq/g of copolymer in a 30% solution in dipropylene glycol, more particularly from 0.05 to 0.2. The weight-average molecular weight (Mw) of the silicone range from 5000 and 1,000,000, more particularly between 10,000 and 200,000. Non-limiting examples include bis-amino PEG/PPG-41/3 aminoethyl PG-propyl dimethicone and PEG-40/PPG-8 methylaminopropyl hydroxypropyl dimethicone copolymer. Commercially available products include the silicones sold under the names SILSOFT A-843 or SILSOFT A+ by Momentive.

g) the alkylamino silicones corresponding to formula (K) below:

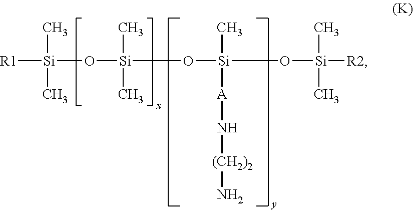

wherein:
x and y are numbers ranging from 1 to 5000; for example x ranges from 10 to 2000 and especially from 100 to 1000; for example y ranges from 1 to 100;
R₁ and R₂, which may be identical or different, such as identical, are linear or branched, saturated or unsaturated alkyl radicals, comprising 6 to 30 carbon atoms, such as from 8 to 24 carbon atoms and especially 12 to 20 carbon atoms; and
A denotes a linear or branched alkylene radical containing from 2 to 8 carbon atoms.

In various exemplary embodiments, A comprises from 3 to 6 carbon atoms, especially 4 carbon atoms, and in certain embodiments, A is branched. Mention may be made of the following divalent radicals: —CH₂CH₂CH₂ and —CH₂CH(CH₃)CH₂—.

Optionally, R₁ and R₂, which may be identical or different, are saturated linear alkyl radicals comprising 6 to 30 carbon atoms, such as from 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R₁ and R₂, which may be identical or different, are chosen from hexadecyl (cetyl) and octadecyl (stearyl) radicals.

In various exemplary embodiments, the silicone of formula (K) is chosen such that:
x ranges from 10 to 2000 and especially from 100 to 1000;
y ranges from 1 to 100;
A comprises 3 to 6 carbon atoms and especially 4 carbon atoms; optionally, A is branched; and more particularly A is chosen from the following divalent radicals: CH₂CH₂CH₂ and —CH₂CH(CH₃)CH₂—; and
R₁ and R₂, which may be identical or different, are linear, saturated alkyl radicals comprising 6 to 30 carbon atoms, such as 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R₁ and R₂, which may be identical or different, being chosen from hexadecyl (cetyl) and octadecyl (stearyl) radicals.

An exemplary amino silicone of formula (K) is bis-cetearylamodimethicone (INCI name), such as the silicone sold under the name SILSOFT AX by Momentive.

Additional useful amino silicones include h) silicone compounds with at least one quaternary ammonium group. Suitable non-limiting examples are quaternium-80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium-80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17. For example, quaternium-80, silicone quaternium-16, silicone quaternium-18, silicone quaternium-15, or mixtures thereof may be chosen.

If present, the amount of the at least one silicone compound that may be included in various embodiments can vary, but in various embodiments the total amount of silicones ranges from about 0.01% to about 10%, based on the total weight of the composition, including all ranges and subranges therebetween. For example, the total amount of silicone compounds may range from about 0.01% to about 8%, from about 0.01% to about 5%, from about 0.01% to about 4%, from about 0.01% to about 3%, from about 0.01% to about 2%, from about 0.1% to about 10%, from about 0.1% to about 8%, from about 0.1% to about 5%, from about 0.1% to about 4%, from about 0.1% to about 3%, from about 0.1% to about 2%, from about 0.5% to about 10%, from about 0.5% to about 8%, from about 0.5% to about 5%, from about 0.5% to about 4%, from about 0.5% to about 3%, from about 0.5% to about 2%, from about 1% to about 10%, from about 1 to about 8%, from about 1% to about 5%, from about 1% to about 4%, from about 1% to about 3%, or from about 1% to about 2%, by weight, relative to the total weight of the composition. In some particular embodiments, the total amount of the at least one silicone may range from about 0.1% to about 5%, or about 0.3% to about 4% by weight, relative to the total weight of the composition.

In one embodiment, the amount of silicone compounds is about 10% or less, such as about 5% or less, such as about 0.2%, about 0.3%, about 0.5%, about 1%, about 1.5%, about 2%, about 2.5%, about 3%, about 3.5%, about 4%, about 4.5%, or about 5%, by weight, relative to the total weight of the composition.

### Fatty Compounds

Compositions according to the disclosure may optionally comprise at least one fatty compound other than silicone compounds and vegetable oils (referred to simply as “fatty compound” or “fatty compounds” herein). Non-limiting examples of suitable fatty compounds include fatty alcohols, fatty esters, fatty ethers, fatty acids, wax, oils, and derivatives thereof.

### Fatty Alcohols

Suitable fatty alcohols, if present, include those having a fatty group with a carbon chain of greater than 8 carbon atoms, such as, for example, from 8 to 50 carbon atoms, 8 to 40 carbon atoms, 8 to 30 carbon atoms, 8 to 22 carbon atoms, 12 to 22 carbon atoms, or 12 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

The fatty alcohol portion may be hydrogenated (for example, stearyl, lauryl, cetyl, cetearyl); however, the fatty alcohol may contain one or more double bonds (for example, oleyl). Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, or mixtures thereof. In some cases, the fatty alcohols comprise at least one of or may be chosen from myristyl alcohol, lauryl alcohol, cetyl alcohol, stearyl alcohol, cetearyl alcohol, isostearyl alcohol, oleyl alcohol, isotridecyl alcohol, or mixtures thereof.

The fatty alcohol may be saturated or unsaturated. Exemplary saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

Exemplary unsaturated liquid fatty alcohol may include in their structure at least one double or triple bond. For example, the fatty alcohols may include several double bonds (such as 2 or 3 double bonds), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

The fatty alcohols may be alkoxylated fatty alcohols, e.g., having about 1 to about 100 moles of an alkylene oxide per mole of alkoxylated fatty alcohol. For example, the alkoxylated fatty alcohols may be alkoxylated with about 1 to about 80 moles, about 2 to about 50, about 5 to about 45 moles, about 10 to about 40 moles, or about 15 to about 35 mores, including all ranges and subranges therebetween, of alkylene oxide per mole of alkoxylated fatty alcohol.

As non-limiting examples of alkoxylated fatty alcohols, steareth (for example, steareth-2, steareth-20, steareth-21, and steareth-100), laureth (for example, laureth-4, laureth-7, and laureth-12), ceteth (for example, ceteth-10 and ceteth-20) and ceteareth (for example, ceteareth-2, ceteareth-7, ceteareth-10, ceteareth-20, and ceteareth-25) are mentioned.

### Fatty Acids

Suitable fatty acids that can be included in the compositions disclosed herein may be include those having from about 10 to about 30 carbon atoms, from about 12 to about 22 carbon atoms, or from about 16 to about 22 carbon atoms. These fatty acids can be straight or branched chain acids and can be saturated or unsaturated. Also included are diacids, triacids, and other multiple acids which meet the carbon number requirement herein. Further included herein are salts of these fatty acids. Nonlimiting examples of fatty acids include myristic acid, lauric acid, palmitic acid, stearic acid, behenic acid, arichidonic acid, oleic acid, isostearic acid, sebacic acid, or mixtures thereof. In some embodiments, the fatty acids may comprise myristic acid, palmitic acid, stearic acid, or mixtures thereof.

### Fatty Esters

The cosmetic compositions may include one or more fatty compound(s) that is a fatty ester. For example, the fatty compound(s) may be chosen from saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10.

In an embodiment, a fatty ester may be chosen from cetyl esters, isopropyl esters, glyceryl (glycerol) esters, dialkyl esters, diesters with octanoic acid and propylene glycol (for example, mixture of the propylene glycol diesters of caprylic and capric acids, propylene glycol dicaprylate/dicaprate, or mixtures thereof, for example may be chosen from cetyl esters, isopropyl esters, glyceryl esters, or mixtures thereof.

Additionally or alternatively, a fatty ester may be chosen from cetyl ester, purcellin oil (cetearyl octanoate), isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, diisostearyl fumarate, or mixtures thereof. Other fatty esters worth mentioning include polyglyceryl-10 oleate, polyglyceryl-10 dioleate, polyglyceryl-6 stearate, polyglyceryl-6 distearate, polyglyceryl-10 stearate, polyglyceryl-10 distearate, polyglyceryl-8 dipalmitate, polyglyceryl-10 dipalmitate, polyglyceryl-10 behenate, and polyglyceryl-12 trilaurate.

In some embodiments, a fatty ester may be chosen from dialkyl carbonates of formula R₁O(C═O)R₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, such as one or more fatty carbonates selected from C14-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, or mixtures thereof.

### Fatty Ethers

The fatty compounds may be chosen from fatty ethers. For example, the cosmetic composition may include olyoxyethylene cetyl/stearyl ether, polyoxyethylene cholesterol ether, polyoxyethylene laurate or dilaurate, polyoxyethylene stearate or distearate, polyoxyethylene lauryl or stearyl ether, dicaprylyl ether, dicetyl ether distearyl ether, dodecyl ether, dilauryl ether, dimyristyl ether, diisononyl ether, or a mixture thereof. Non-limiting examples of suitable polyoxyethylene fatty ethers include, but are not limited to, polyoxyethylene cetyl/stearyl ether, polyoxyethylene cholesterol ether, polyoxyethylene laurate or dilaurate, polyoxyethylene stearate or distearate, polyoxyethylene lauryl or stearyl ether, and mixtures thereof, wherein the polyoxyethylene head group ranges from about 2 to about 100 groups. In certain embodiments, the polyoxyethylene fatty ethers include polyoxyethylene stearyl ether, polyoxyethylene myristyl ether, polyoxyethylene lauryl ether having from about 3 to about 10 oxyethylene units and mixtures thereof.

### Derivatives

When fatty compounds are mentioned, derivatives of fatty compounds are intended to be included, whether or not so stated, unless expressly indicated otherwise.

Fatty alcohol derivatives include alkyl ethers of fatty alcohols, alkoxylated fatty alcohols, alkyl ethers of alkoxylated fatty alcohols, esters of fatty alcohols or mixtures thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcohols, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; or mixtures thereof.

Non-limiting examples of polyglycerol esters of fatty acids include those of the following formula (III):

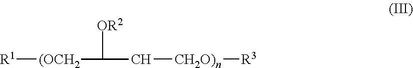

wherein:
the average value of n is about 3; and
R¹, R², and R³, which may be identical or different, are independently chosen from a fatty acid moiety or hydrogen, provided that at least one of R¹, R², and R³ is a fatty acid moiety.

For instance, R¹, R², and R³ may be saturated or unsaturated, linear or branched, and have a length of C₁-C₄₀, C₁-C₃₀, C₁-C₂₅, C₁-C₂₀, C₁-C₁₆, or C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, or mixtures thereof.

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, or mixtures thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, or mixtures thereof. Preferred for use herein are glycerol monostearate, 12-hydroxy stearic acid, or mixtures thereof.

In some cases, the one or more fatty compounds may be one or more high melting point fatty compounds. A high melting point fatty compound is a fatty compound having a melting point of 25° C. Even higher melting point fatty compounds may also be used, for example, fatty compounds having a melting point of 40° C. or higher, 45° C. or higher, or 50° C. or higher. The high melting point fatty compound may be selected from fatty acids, fatty alcohol derivatives, fatty acid derivatives, or mixtures thereof. Nonlimiting examples of the high melting point compounds are found in the International Cosmetic Ingredient Dictionary, Sixteenth Edition, 2016, which is incorporated by reference herein in its entirety.

In various embodiments, the total amount of fatty compounds in compositions disclosed herein may range from about 0.01% to about 30%, including all subranges therebetween, such as from about 0.1% to about 30%, about 0.1% to about 25%, about 0.1% to about 20%, about 0.1% to about 15%, about 0.1% to about 10%, about 0.1% to about 5%, about 0.5% to about 30%, about 0.5% to about 25%, about 0.5% to about 20%, about 0.5% to about 15%, about 0.5% to about 10%, about 0.5% to about 5%, about 1% to about 30%, about 1% to about 25%, about 1% to about 20%, about 1% to about 15%, about 1% to about 10%, about 1% to about 8%, about 1% to about 6%, about 1% to about 5%, about 2% to about 30%, about 2% to about 25%, about 2% to about 20%, about 2% to about 15%, about 2% to about 10%, about 2% to about 8%, about 2% to about 6%, about 2% to about 5%, about 3% to about 30%, about 3% to about 25%, about 3% to about 20%, about 3% to about 15%, about 3% to about 10%, about 3% to about 8%, about 3% to about 6%, about 3% to about 5%, about 4% to about 30%, about 4% to about 25%, about 4% to about 20%, about 4% to about 15%, about 4% to about 10%, about 4% to about 8%, about 4% to about 6%, about 4% to about 5%, about 5% to about 30%, about 5% to about 25%, about 5% to about 20%, about 5% to about 15%, about 5% to about 10%, about 5% to about 8%, or about 5% to about 6% by weight, relative to the total weight of the composition.

### Vegetable Oils

In various embodiments, compositions according to the disclosure may further comprise at least one vegetable oil. Vegetable oils according to the disclosure are oils derived from a plant, for example, oils from seeds or fruits. A high level of vegetable oil, such as an amount of about 1% or higher, is not typically included in traditional emulsion cosmetic compositions because oils at this level may negatively impact the stability of the emulsion cosmetic composition. However, it has been found that including vegetable oil in the compositions according to the disclosure, when combined with other ingredients disclosed herein, results in a stable emulsion composition that provides beneficial treatment properties to hair, even at a relatively high concentration.

Vegetable oils that may be used according to the disclosure include, but are not limited to, coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, walnut oil, and mixtures thereof. In various embodiments, compositions according to the disclosure comprise coconut oil. In other embodiments, compositions according to the disclosure comprise avocado oil, sunflower oil, and/or camelina sativa seed oil. In further embodiments, compositions according to the disclosure are substantially free of vegetable oils other than coconut oil. Thus, in various embodiments, the at least one vegetable oil may comprise, consist essentially of, or consist of coconut oil.

The total amount of vegetable oil that may be included in various embodiments may range from about 0.1% to about 15%, including all subranges therebetween, such as from about 0.5% to about 12%, about 0.5% to about 10%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1.5%, or about 0.5% to about 1%, based on the total weight of the composition. In certain embodiments, vegetable oil is present in an amount of about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, or about 7% by weight, relative to the total weight of the composition. In some embodiments, the compositions disclosed herein comprise coconut oil, avocado oil, and/or sunflower oil in a total amount ranging from about 0.05% to about 5%, such as about 0.05% to about 2.5% by weight, relative to the total weight of the composition.

### Waxes

In some instances, the non-silicone fatty compounds may include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax (Helianthus annuus), acacia decurrents flower wax, or a mixture thereof.

In one embodiment, the composition includes waxes. Mention may be made, among the waxes capable of being used as non-silicone fatty compounds, of animal waxes, such as beeswax; vegetable waxes, such as sunflower seed (Helianthus annuus), carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis.

### Non-Silicone Oils

In some instance, the non-silicone fatty compounds include one or more non-silicone oils other than vegetable oils described above. The term “oil” as used herein describes any material which is substantially insoluble in water. Suitable non-silicone oils include, but are not limited to, natural oils; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Suitable low viscosity oils have a viscosity of 5-100 mPas at 25° C. and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity ranging from 200-1,000,000, such as from 100,000-250,000, mPas at 25° C. Such oils include lanolin and lanolin derivatives, triisocetyl citrate, sorbitan sesquioleate, C₁₀-C₁₈ triglycerides, caprylic/capric/triglycerides, glyceryl triacetyl hydroxystearate, glyceryl triacetyl ricinoleate, glyceryl trioctanoate, mink oil, illipe butter, tallow, tricaprin, trihydroxystearin, triisostearin, trilaurin, trilinolein, trimyristin, triolein, tripalmitin, tristearin, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

### Polyols

The compositions according to the disclosure may optionally comprise at least one polyol. In certain embodiments, compositions comprise at least two polyols. As used herein, the term “polyol” refers to an organic molecule comprising at least two free hydroxyl groups. The polyols may be liquid at ambient temperature (25° C.). Suitable polyols that may be comprised in the compositions disclosed herein may be glycols or compounds with numerous hydroxyl groups. In some cases, the at least one polyol is chosen from polyols having 2-32 carbon atoms, i.e., C2-C32 polyols, such as from 3 to 16 carbon atoms, or from 3 to 12 carbon atoms.

By way of example, the at least one polyol, when present, may be chosen from ethylene glycol, propylene glycol, butylene glycol, hexylene glycol, glycerin, diglycerin, diethylene glycol, and dipropylene glycol, polyethylene glycols, or mixtures thereof. In some cases, the compositions include propylene glycol. In some further cases, the compositions include one or both of propylene glycol and butylene glycol. Additionally, in some cases, the composition comprises propylene glycol, and optionally at least one polyol other than propylene glycol.

Non-limiting examples of polyols that may, optionally, be included in the compositions according to the disclosure include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and/or a mixture thereof.

In some embodiments, the at least one polyol that is optionally included is chosen from glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol, or mixtures thereof. In some cases, a composition according to the disclosure may include at least one polyol chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, or a mixture thereof.

If present, the total amount of polyols may vary but typically ranges from about 0.1% to about 15%, including all subranges therebetween, such as from about 0.1% to about 15%, from about 0.1% to about 12%, from about 0.1% to about 10%, from about 0.1% to about 8%, from about 0.1% to about 5%, from about 0.1% to about 4%, from about 0.1% to about 3%, from about 0.1% to about 2%, from about 0.1% to about 1.5%, from about 0.1% to about 1%, from about 0.5% to about 15%, from about 0.5% to about 12%, from about 0.5% to about 10%, from about 0.5% to about 8%, from about 0.5% to about 5%, from about 0.5% to about 4%, from about 0.5% to about 3%, from about 0.5% to about 2%, from about 0.5% to about 1.5%, from about 0.5% to about 1%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, or from about 1.5% to about 2% by weight, relative to the total weight of the composition. For example, the at least one polyol may be present in an amount ranging from about 1 to about 9%, such as about 1.5% to about 8.5% by weight, relative to the total weight of the composition.

### Cationic Surfactants

Compositions according to the disclosure may optionally include at least one cationic surfactant.

In certain embodiments, compositions disclosed herein may include at least one cationic surfactant chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamidopropyidiethylamine, arachidamidoethyldiethylamine, arachidamidoethyldimethylamine, or mixtures thereof.

Additional, non-limiting examples of cationic surfactants include behenalkonium chloride, benzethonium chloride, cetylpyridinium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylamino-ethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procaine-hydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, and mixtures thereof.

In some embodiment, the composition may optionally comprise at least one cationic surfactant chosen from polyoxyalkylenated, primary, secondary or tertiary fatty amines, or salts thereof, and quaternary ammonium salts, or mixtures thereof. In some cases, it is useful to use salts such as chloride salts of the quaternary ammonium compounds.

The fatty amines generally comprise at least one C₈-C₃₀ hydrocarbon-based chain. For example, quaternary ammonium salts, which may be incorporated in certain instances, include those corresponding to the following general formula (IV):

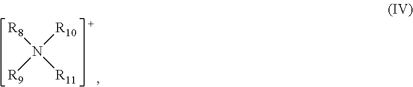

wherein the groups R₈ to R₁₁, which may be identical or different, independently represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms, such as from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts having a structure in accordance with the above general formula (IV), those that are preferred are, on the one hand, tetraalkylammonium salts, for instance dialkyldimethylammonium or alkyltrimethylammonium salts in which the alkyl group contains approximately from 12 to 22 carbon atoms, such as behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltri-methylammonium salts, stearamidopropyltrimethylammonium salts and stearamidopropyldimethyl-cetearylammonium salts.

Examples of quaternary ammonium salt of imidazoline, which may be incorporated in certain instances, include those having a structure according to the general formula (V) provided below:

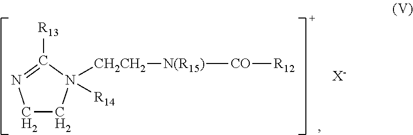

in which R12 represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups may comprise, respectively, from 1 to 20 carbon atoms and from 6 to 30 carbon atoms. R₁₂ and R₁₃ may denote a mixture of alkenyl or alkyl groups containing from 12 to 21 carbon atoms, derived for example from tallow fatty acids, R₁₄ optionally denotes a methyl group, and R₁₅ optionally denotes a hydrogen atom. Such a product is sold, for example, under the name REWOQUAT W 75 by the company Rewo.

Examples of quaternary diammonium or triammonium salt, which may be incorporated in certain instances, include those having a structure in accordance with the following general formula (VI):

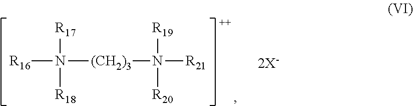

in which R₁₆ denotes an alkyl radical comprising approximately from 16 to 30 carbon atoms, which is optionally hydroxylated and/or interrupted with one or more oxygen atoms; R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms; and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75).

Examples of cationic/cationizable surfactants, which may be incorporated in certain instances, include those having a structure in accordance with the general formula (VII) provided below:

R4-A-R5-B (VII)

wherein R4 is a saturated or unsaturated, straight or branched alkyl chain with 8 to 24 carbon atoms, R5 is a straight or branched alkyl chain with 1 to 4 carbon atoms, A is selected from:

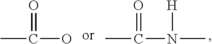

and B is selected from:

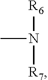

wherein R₆ and R₇, which are the same or different, are chosen from hydrogen or alkyl chains with 1 to 4 carbon atoms, hydroxyl alkyl chains with 1 to 4 carbon atoms, or di-hydroxyl alkyl chain with 2 to 4 carbon atoms, or

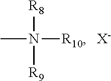

wherein R₈ and R₉, which are the same or different, are chosen from alkyl chains with 1 to 4 carbon atoms, hydroxyl alkyl chains with 1 to 4 carbon atoms, or di-hydroxyl alkyl chains with 2 to 4 carbon atoms; R₁₀ is chosen from alkyl chains with 1 to 4 carbon atoms, hydroxyl alkyl chains with 1 to 4 carbon atoms, or di-hydroxyl alkyl chains with 2 to 4 carbon atoms.

In some instances, R₄ in formula (VII) is a saturated or unsaturated, straight or branched alkyl chain with 10 to 24 carbon atoms, e.g. 12 to 22 carbon atoms, and R₅ is a straight or branched alkyl group with 1 to 4 carbon atoms, and A, B, and R₆ to R₁₀ are same as above.

Non-limiting suitable examples of cationic surfactants are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl butylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl butylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, stearamidopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants may be chosen from fatty alkylamines, such as fatty dialkylamines. Non-limiting examples include dimethyl lauramine, dimethyl behenamide, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, stearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamidopropyidiethylamine, arachidamidoethyldiethylamine, arachidamidoethyldimethylamine, brassicamidopropyldimethylamine, lauramidopropyl dimethylamine, myristamidopropyl dimethylamine, dilinoleamidopropyl dimethylamine, and palmitamidopropyl dimethylamine.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, or mixtures thereof. In particular, lactic acid or tartaric acid or mixtures thereof are useful, especially in combination with fatty dimethylamines such as, for example, stearamidopropyl dimethylamine.

In an embodiment, a composition disclosed herein may be formulated with a cationic surfactant chosen from behentrimonium chloride, cetrimonium chloride, behentrimonium methosulfate, or mixtures thereof.

The compositions disclosed herein may be formulated such that the two or more cationic surfactants are associated with the same or different balancing anionic ions. For example, at least one of the two or more cationic surfactants may have a chloride ion and/or a sulfate ion. In some instances, the two or more cationic surfactants comprise cetrimonium chloride and one or both of behentrimonium methosulfate and behentrimonium chloride. In further instances, the two or more cationic surfactants comprise behentrimonium chloride and one or both of behentrimonium methosulfate and cetrimonium chloride.

In yet another instance, the at least one cationic surfactant, when present, is chosen from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyldiethylamine, arachidamidoethyldimethylamine, or mixtures thereof.

In various embodiments, the total amount of cationic surfactants, when present, may range from about 0.1% to about 10% by weight, including all subranges therebetween, such as from about 0.1% to about 10%, from about 0.1% to about 8%, from about 0.1% to about 6%, from about 0.1% to about 4%, from about 0.1% to about 3%, from about 0.1% to about 2.5%, from about 0.1% to about 2%, from about 0.1% to about 1.5%, from about 0.1% to about 1%, from about 0.5% to about 10%, from about 0.5% to about 8%, from about 0.5% to about 6%, from about 0.5% to about 4%, from about 0.5% to about 3%, from about 0.5% to about 2.5%, from about 0.5% to about 2%, from about 0.5% to about 1.5%, from about 0.5% to about 1%, from about 1% to about 10%, from about 1% to about 8%, from about 1% to about 6%, from about 1% to about 4%, from about 1% to about 3%, from about 1% to about 2.5%, or from about 1% to about 2% by weight, based on the total weight of the composition.

### Solvents

Compositions according to the disclosure comprise a cosmetically acceptable solvent. The solvent may comprise water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises one or more non-aqueous solvents, other than or in addition to ingredients discussed above. For example, C₁₋₄ alcohols, organic solvents, fatty alcohols, fatty ethers, fatty esters, polyols other than those described above, glycols, vegetable oils, mineral oils, liposomes, laminar lipid materials, or any a mixture thereof. Non-limiting examples of solvents which may be used include alkane polyols such as 1,2,6-hexanetriol, trimethylolpropane, ethylene glycol, propylene glycol, diethylene glycol, hexylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbitol, sorbitan, acetine, diacetine, triacetine, sulfolane, or mixtures thereof. At least in some embodiments, the compositions contain water and at least one, for example two or more, additional solvent chosen from caprylyl glycol, hexylene glycol, ethylhexylglycerin, glycerin, or mixtures thereof.

The solvent may be present in the composition in an amount ranging from about 20% to about 90% by weight, relative to the total weight of the composition, including all ranges and subranges therebetween. For example, in one embodiment, the total amount of solvent may be about 20% to about 85%, about 25% to about 85%, about 30% to about 85%, about 40% to about 85%, about 45% to 85%, or about 50% to 80% by weight, relative to the total weight of the composition.

In certain embodiments, the solvent is primarily comprised of water, such as from about 20% to about 85%, about 20% to about 80%, about 20% to about 75%, about 20% to about 70%, about 20% to about 60%, about 20% to about 50%, about 20% to about 40%; from about 25% to about 90%%, about 25% to about 85%, about 25% to about 80%, about 25% to about 75%, about 25% to about 70%, about 25% to about 60%, about 25% to about 50%, about 25% to about 40%; from about 30% to about 90%%, about 30% to about 85%, about 30% to about 80%, about 30% to about 75%, about 30% to about 70%, about 30% to about 60%, about 30% to about 50%; from about 35% to about 90%%, about 35% to about 85%, about 35% to about 80%, about 35% to about 75%, about 35% to about 70%, about 35% to about 60%, about 35% to about 50%; from 40% to about 90%%, about 40% to about 85%, about 40% to about 80%, about 40% to about 75%, about 40% to about 70%, or about 40% to about 60% by weight, relative to the total weight of the composition.

### Additional Components

Compositions according to the disclosure may optionally comprise one or more additional components suitable for use in such compositions. Non-limiting examples of such additional components are provided below.

### Thickening Agents

The compositions described herein may, optionally, include at least one thickening agent. “Thickening agents” as used here may be referred to as “thickeners” or “viscosity modifying agents.” Thickening agents are typically included to increase the viscosity of the cosmetic compositions. Nonetheless, in some instances, certain thickening agents provide additional, surprising benefits to the compositions.

In some embodiments, thickening agents are water-soluble, and increase the viscosity of water or form an aqueous gel when ingredients according to the disclosure are dispersed/dissolved in water to formulate the compositions. The aqueous solution may be heated and cooled, or neutralized, for forming the gel, if necessary. The thickener may be dispersed/dissolved in an aqueous solvent that is soluble in water, e.g., ethyl alcohol when it is dispersed/dissolved in water.

As non-limiting examples, thickening agents may be chosen from xanthan gum, guar gum, biosaccharide gum, cellulose, acacia seneca gum, sclerotium gum, agarose, pechtin, gellan gum, hyaluronic acid, or mixtures thereof. Additionally, the at least one thickening agent may include polymeric thickening agents chosen from ammonium polyacryloyldimethyl taurate, ammonium acryloyldimethyltaurate/VP copolymer, sodium polyacrylate, acrylates copolymers, polyacrylamide, carbomer, acrylates/C10-30 alkyl acrylate crosspolymer, or mixtures thereof. In some cases, the composition may optionally include ammonium polyacryloyldimethyl taurate and/or sodium polyacrylate. Suitable thickening agents may be found in U.S. patent application Ser. No. 16/731,654, which is incorporated herein, in its entirety for all purposes.

According to some embodiments, non-limiting examples of thickening agents may include polyacrylate crosspolymers or crosslinked polyacrylate polymers, cationic acrylate copolymers, anionic acrylic or carboxylic acid polymers, polyacrylamide polymers, polysaccharides such as cellulose derivatives, gums, polyquaterniums, vinylpyrrolidone homopolymers/copolymers, C8-24 hydroxyl substituted aliphatic acid, C8-24 conjugated aliphatic acid, sugar fatty esters, polyglyceryl esters, or mixtures thereof. Particular types of thickening agents that may be mentioned include the following:

(1) Carboxylic acid or carboxylate based homopolymer or co-polymer, which can be linear or crosslinked:

These polymers contain one or more monomers derived from acrylic acid, substituted acrylic acids, and salts and esters of these acrylic acids (acrylates) and the substituted acrylic acids. Commercially available polymers include those sold under the trade names CARBOPOL, ACRYSOL, POLYGEL, SOKALAN, CARBOPOL ULTREZ, and POLYGEL. Examples of commercially available carboxylic acid polymers include the carbomers, which are homopolymers of acrylic acid crosslinked with allyl ethers of sucrose or pentaerytritol. The carbomers are available as the CARBOPOL 900 series from B.F. Goodrich (e.g., CARBOPOL 954). In addition, other suitable carboxylic acid polymeric agents include ULTREZ 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as CARBOPOL 1342, CARBOPOL 1382, PEMULEN TR-1, and PEMULEN TR-2, from B.F. Goodrich.

Other suitable carboxylic acid or carboxylate polymeric agents include copolymers of acrylic acid and alkyl C5-C10 acrylate, copolymers of acrylic acid and maleic anhydride, and polyacrylate crosspolymer-6. Polyacrylate Crosspolymer-6 is available in the raw material known as SEPIMAX ZEN from Seppic.

Another suitable carboxylic acid or carboxylate polymeric agent includes acrylamidopropyltrimonium chloride/acrylates copolymer, a cationic acrylates copolymer (or a quaternary ammonium compound), available as a raw material known under the tradename of SIMULQUAT HC 305 from Seppic.

In certain embodiments, the carboxylic acid or carboxylate polymer thickening agents useful herein are those selected from carbomers, acrylates/C10-C30 alkyl acrylate crosspolymers, polyacrylate crosspolymer-6, acrylamidopropyltrimonium chloride/acrylates copolymer, or mixtures thereof.

(2) Polyquaternium Compounds:

Non-limiting examples, include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, or mixtures thereof.

(3) Celluloses:

Non-limiting examples of celluloses include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, or mixtures thereof. In some instances, the cellulose is selected from water-soluble cellulose derivatives (for example, carboxymethyl cellulose, methyl cellulose, methylhydroxypropyl cellulose, hydroxyethyl cellulose, hydroxypropyl cellulose, cellulose sulfate sodium salt). Furthermore, in some instance, the cellulose is hydroxypropylcellulose (HPC).

(4) Polyvinylpyrrolidone (PVP) and Co-Polymers:

Non-limiting examples include Polyvinylpyrrolidone (PVP), Polyvinylpyrrolidone (PVP)/vinyl acetate copolymer (PVP/VA copolymer), polyvinylpyrrolidone (PVP)/eicosene copolymer, PVP/hexadecene copolymer, etc. Commercially available polyvinylpyrrolidone includes LUVISKOL K30, K85, K90 available from BASF. Commercially available copolymers of vinylpyrrolidone and vinylacetate include LUVISKOL VA37, VA64 available from BASF; copolymers of vinylpyrrolidone, methacrylamide, and vinylimidazole (INCI: VP/Methacrylamide/Vinyl Imidazole Copolymer) is commercially available as LUVISET from BASF. In some instances, PVP and PVP/VA copolymer are preferred.

(5) Sucrose esters:

Non-limiting examples include sucrose palmitate, sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate, and mixtures thereof.

(6) Polyglyceryl esters:

Non-limiting polyglycerol esters of fatty acids (polyglyceryl esters) include those of the following formula (VIII):

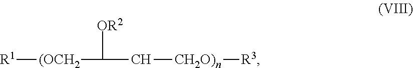

wherein n is from 2 to 20 or from 2 to 10 or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, linear or branched, and have a length of C₁-C₄₀, C₁-C₃₀, C₁-C₂₅, or C₁-C₂₀, C₁-C₁₆, or C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.

(7) Gums:

Non-limiting examples of gums include gum arabic, tragacanth gum, karaya gum, guar gum, gellan gum, tara gum, locust bean gum, tamarind gum, xanthan gum, locust bean gum, Seneca gum, sclerotium gum, gellan gum, etc.

In various embodiments, the total amount of thickening agents, when present, may range from about 0.01% to about 10% by weight, including all subranges therebetween, such as from about 0.01% to about 9%, from about 0.01% to about 8%, from about 0.01% to about 6%, from about 0.01% to about 4%, from about 0.01% to about 3%, from about 0.01% to about 2.5%, from about 0.01% to about 2%, from about 0.01% to about 1.5%, from about 0.01% to about 1%, from about 0.05% to about 10%, from about 0.05% to about 8%, from about 0.05% to about 6%, from about 0.05% to about 4%, from about 0.05% to about 3%, from about 0.05% to about 2.5%, from about 0.05% to about 2%, from about 0.05% to about 1.5%, from about 0.05% to about 1%, from about 0.1% to about 10%, from about 0.1% to about 8%, from about 0.1% to about 6%, from about 0.1% to about 4%, from about 0.1% to about 3%, from about 0.1% to about 2.5%, from about 0.1% to about 2%, from about 0.1% to about 1.5%, or from about 0.1% to about 1% by weight, based on the total weight of the composition. For example, compositions according to the disclosure may comprise a total amount of thickening agents ranging from about 0.1% to about 0.9%, about 0.2% to about 0.8%, about 0.3% to about 0.7%, or about 0.4% to about 0.6% by weight, based on the total weight of the composition.

### Hydrating Agents

In some embodiments, compositions according to the disclosure comprise at least one hydrating agent. Hydrating agents that can be used include both synthetic and natural hydrating agents. Nonlimiting examples of hydrating agents include squalane, sucrose, triacetin, xylitol, maltitol, sorbitol, pentaerythritol, inositol, pyrrolidone carboxylic acid, lactic acid, lithium chloride, acetamide MEA, sodium lactate, dicyanamide, hyaluronic acid, aloe vera, honey, and seaweed extract. Mixtures of two or more hydrating agents may also be chosen. In at least one embodiment, honey is particularly useful as a hydrating agent.

The compositions may comprise one or more hydrating agents in an amount useful to provide hydration benefits to the hair. For example, the compositions may comprise a total amount of hydrating agents ranging from about 0.001% to about 10%, such as about 0.01% to about 8% by weight, relative to the total weight of the composition. In some embodiments, the compositions comprise a total amount of hydrating agents ranging from about 0.001% to about 1%, such as about 0.01% to about 0.5%, or about 0.01% to about 0.1% by weight, relative to the total weight of the composition.

### pH Adjusters

The compositions may include one or more pH adjusters to increase or decrease the overall pH of the composition. For example, one or more acids may be included to decrease the pH of the cosmetic composition. Examples of suitable acids for decreasing the pH of the compositions may include one or more acids, such as acetic acid and the like. The cosmetic composition may include one or more bases, such as sodium hydroxide, potassium hydroxide and the like, to increase the pH of the cosmetic composition. Additional or alternative acids and bases that are suitable for adjusting the pH of the compositions are readily known to one of ordinary skill in the art.

The amount of the pH adjuster in the compositions may be based on the desired pH of the final composition and/or product for improving curl definition, curl regularity, and/or curl elongation. For example, the total amount of the pH adjuster may range from about 0.0001% to about 15%, based on the total weight of the cosmetic composition. In some instances, the total amount of pH adjuster can range from about 0.005% to about 10%, about 0.01% to about 5%, about 0.1% to about 1%, or about 0.1% to about 0.5% by weight, including ranges and sub-ranges therebetween, based on the total weight of the cosmetic composition. For instance, in one embodiment, the pH adjuster is about 0.1M of sodium hydrate (NaOH).

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), or mixtures thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, or mixtures thereof.

The total amount of preservatives, when present, may vary. In some cases, the total amount preservatives can range about 0.01% to about 5%, about 0.01% to about 4%, about 0.15% to about 1%, or about 1% to about 3%, by weight, relative to the total weight of the composition.

### Auxiliary Components

Compositions according to the disclosure may optionally comprise any auxiliary component suitable for use in such compositions. Such components may include, but are not limited to, dyes/pigments for adding color to the composition, moisturizing agents, fatty substances, thickeners other than those previously described, fillers, structuring agents, shine agents, antioxidants or reducing agents, penetrants, sequestrants, fragrances, buffers, dispersants, plant extracts, such as apricot seed powder, opacifiers, emulsifiers, sunscreen agents, vitamins, amino acids (e.g. glycine), and antistatic agents.

Optional auxiliary components may be present in an amount ranging up to about 15%, such as from about 0.001% to about 10%, from about 0.01% to about 5%, or from about 0.1% to about 3% by weight, relative to the total weight of the composition.

In various embodiments, the compositions are acidic and may have a pH of less than about 10, for example less than about 7, such as a pH ranging from about 1.5 to about 6.8, from about 2 to about 6.5, from about 2.5 to about 6, from about 3 to about 6.5, from about 3 to about 6, from about 3 to about 5.5, from about 3 to about 5, from about 3 to about 4, from about 3.5 to about 6.5, from about 3.5 to about 6, from about 3.5 to about 5.5, from about 3.5 to about 5, from about 3.5 to about 4.5, or from about 3.5 to about 4, including all ranges and subranges therebetween. In some embodiments, the pH of the composition ranges from about 3 to about 6.5. In other embodiments, the pH of the composition ranges from about 3 to about 4.

Compositions according to the disclosure may be leave-in compositions or rinse-off compositions, and are typically in the form of a cream, paste, or lotion, but may also be in the form of a serum, a gel, a gel cream, or the like. In some embodiments, the compositions may be a hair treatment or conditioner product, a hair styling product, or a product that styles hair while providing treatment and/or conditioning benefits. In at least one embodiment, the compositions are hair styling compositions that provide conditioning benefits, but are not, per se, hair conditioner compositions.

The viscosity of the compositions according to the disclosure is not limited, but by way of example only, in certain embodiments the viscosity may range from about 450 cPs to about 6900 cPs, including all ranges and subranges therebetween, when measured with a viscometer (#4 spindle) at 25° C.

II. Kits

The disclosure also relates to kits comprising the compositions described herein. The kits in various embodiments may comprise at least one container suitable for containing and/or dispensing the compositions described herein for applying to the hair. In some other embodiments, a kit may comprise a first container containing a composition according to the disclosure, and at least one additional container comprising a composition according to the disclosure or a composition not according to the disclosure, such as, for example, a shampoo, a conditioner, a hair mask, or a hair styling composition.

III. Methods

Compositions described herein surprisingly provide a variety of desirable benefits such as curl definition, curl regularity, curl hold, and/or elongation (even at high humidity conditions and/or over a period of time), as well as other sensory benefits, for example, moisture, smoothness, softness, good bounce, volume control, and/or good shine, to curly or waving hair. As such, the compositions are useful in methods of styling (which includes shaping) hair, as well as methods of treating or caring for hair, conditioning hair, and/or imparting one or more sensory benefits describe above to the hair, e.g., curly hair.

Therefore, the disclosure also relates to methods for treating, caring for, and/or styling hair, e.g., curly hair, for example, by improving curl definition, curl regularity, and/or elongation of curly hair, with compositions described herein.

In various embodiments, methods according to the disclosure are methods for styling hair, in particular curly hair, and comprise applying a composition according to the disclosure to the hair. In other embodiments, the methods comprise methods for caring for or treating hair, e.g. by improving the smoothness, softness, sheen, etc. of hair, and comprise applying a composition according to the disclosure to the hair. In still other embodiments, the methods comprise methods for styling and caring for/treating hair simultaneously.

In some other embodiments, the methods are methods for improving definition and/or regularity of curls and/or curl hold, and/or reducing frizziness of hair, by providing lasting curl definition and curl regularity of the hair, e.g. providing lasting curl definition and/or curl regularity for 1 hour or more, for 4 hours or more, for 8 hours or more, for 12 hours or more, for 16 hours or more, for 24 hours or more, or for 72 hours or more, after application of a composition according to the disclosure.

In some embodiments, the methods are methods for increasing the perceived length of hair, reducing shrinkage of curly hair, and/or reducing frizziness of hair by providing lasting curl elongation and/or curl definition of the hair, e.g. providing lasting curl elongation and/or curl definition for 1 hour or more, for 4 hours or more, for 8 hours or more, for 12 hours or more, for 16 hours or more, for 24 hours or more, or for 72 hours or more, after application of a composition according to the disclosure.

The methods according to the disclosure may vary but typically include applying an effective amount of a composition disclosed herein to hair, such as curly hair. The compositions may be generally applied to hair that is wet, but may also be applied to the hair that is dry, damp, or moist. As used herein, the term “effective amount” refers to an amount sufficient to provide a desired curl definition, regularity, and/or elongation effect to the hair, depending on the degree of curliness, the length, the volume, and the texture of the hair. In general, from about 0.1 grams to about 50 grams of product is applied to the hair, depending on the specific product formulation, hair length, hair volume, and hair style type. In some embodiments, about 0.1 grams (g) of the composition per gram of hair is applied to the hair. In some embodiments, about 0.5 grams (g) of the composition per gram of hair is applied to the hair. The composition applied to the hair may be distributed through the hair as desired, such as substantially evenly or uniformly massaged throughout the hair by combing through with fingers or a means such as a comb or the like.

After the application of the composition to the hair, the hair may then be dried, for example air-dried or dried with a hair dryer. The composition may be allowed to remain on the hair as a leave-in product for any period of time as needed, for example, from about a few seconds (e.g., 1, 3, 5, or 10 seconds) to about 10, 20, or 30 minutes, or longer, such as a few hours or a few days, or until the next washing or rinsing of the hair. In some embodiments, the compositions applied to the hair are allowed to stay on the hair to be air-dried overnight before styling.

In some embodiments, the compositions are rinse-off compositions, meaning they are intended to be left on the hair for a period of time to impart benefits, but are typically rinsed off before the hair is subsequently dried and/or styled. When the compositions are used as rinse-off compositions, the compositions are usually allowed to stay on hair for a period of time, for example at least a few seconds, such as about 30 seconds, or at least a few minutes, such as at least 5 minutes, before rinsing. Therefore, in various methods according to the disclosure, the methods comprise a step of rinsing the composition from the hair.

In some embodiments, before application of the composition to the hair, the hair may be first cleansed with a commercially available shampoo or be rinsed with water. The hair may be further conditioned and/or rinsed after shampooing and before application of the compositions disclosed herein. After the hair is shampooed, conditioned, and/or rinsed, the composition is then applied to the washed or rinsed hair when the hair is wet, damp, or moist. In some other embodiments, before applying the composition to dry hair, the hair can optionally be moistened, damped, or wetted by water spray or using a wet towel, or by applying other treatment compositions that make the hair moist, damp, or wet.

Alternatively, in some embodiments, the compositions according to the disclosure may be first applied to dry hair, and then water or a cosmetically acceptable compositions are applied to the hair to moist, damp, or wet the hair when the compositions according to the disclosure are remained thereon. The hair that has been applied with the compositions disclose herein may be air-dried without further treatment of the hair, including rinsing or washing the hair.

In some embodiments, a composition of the disclosure may be applied to the hair and left on the hair for holding curls or other hair styles. In some embodiments, the hair is styled during the application of the compositions, or when the compositions are allowed to stay on the hair and the hair is still wet. In some embodiments, the hair is allowed to air dry after application of the composition and is subsequently styled or shaped with no heat, such as from an iron, blow dryer, or the like, being applied to the hair. Therefore, some methods comprise styling and/or caring for/treating hair in the absence or substantial absence of heat or a heating step. Styling or shaping the hair may involve the use of devices on hair such as a brush, a comb or running the fingers of the hand through the hair. Optionally, styling or shaping can be done using twisting out techniques. In this situation, the composition disclosed herein may be applied to the hair before or during performing twist out, where the hair is twist and then let go to create curls or other hair styles. In some embodiments, the hair treated with the compositions may be twisted within 30 minutes, or within 20 minutes, while the hair is wet or damp.

In some embodiments, methods according to the disclosure do not require shaping or styling the hair before, during, and/or after applying the compositions with a heating device. A tension to the hair is generally not needed. As such, in some embodiments, before, during, or after applying the compositions, the hair is not stretched by a mechanical means.

In another embodiment, the methods may include styling or shaping the hair with a heating device, e.g., hair dryer, before, during, or after the compositions are applied to the hair.

Methods according to the disclosure generally do not require the use of a reducing agent, including a base, or heating the hair for stretching the hair curls or elongating the hair curls. As such, in at least some embodiments, the method does not involve using a reducing agent, or a base, and/or does not require or include a step of heating the hair after the composition is applied thereto the hair in order to achieve the desired results.

In some embodiments, a composition disclosed herein is used alone to treat the hair, without the use of other hair treatment compositions. In some other embodiments, the compositions may be applied to the hair before, during, or after another hair treatment or styling composition, such as (e.g. a conditioner, a mask, a cream, a lotion, a gel, a coloring composition, etc.), is applied to the hair. As such, a composition disclosed herein may be applied to the hair and layered with another composition that is also applied to the hair.

In various embodiments, the methods of treating hair with the compositions according to the disclosure, impart various long-lasting benefits described above to the hair, relative to hair not having been treated with a composition according to the disclosure. The term “last” or “long-lasting,” as used herein, means that the elongation effect and other benefits imparted to the hair may remain a desired length of time, such as a few hours, a few days, or until the hair is rinsed or washed.

Having described the many embodiments of the present invention in detail, it will be apparent that modifications and variations are possible without departing from the scope of the disclosure defined in the appended claims. Furthermore, it should be appreciated that all examples in the disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, 2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

“Active material” as used herein with respect to the percent amount of an ingredient or raw material, refers to 100% activity of the ingredient or raw material.

As used herein, a “leave-in” or “leave-on” composition or product refers to a composition such as a hair-treatment or hair-styling composition that is not rinsed and/or washed away with water or acceptable solvent after the application of the composition onto the hair; instead, the composition is allowed to remain on the hair for a period of time as desired, such from about 1 hour, 2 hours, 3 hours, 4 hours, up to 8 hours, overnight, or as long as needed, until next time of washing or rinsing the hair.

All amounts given herein are relative to the amount of active material, unless otherwise indicated.

All percentages, parts and ratios herein are based upon the total weight of the compositions of the disclosure, unless otherwise indicated.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

As used herein, the terms “applying a composition onto keratin fibers” and “applying a composition onto hair” and variations of these phrases are intended to mean contacting the keratin fibers including hair, with at least one of the compositions of the disclosure, in any manner. It may also mean contacting the keratin fibers in an effective amount of the composition.

As used herein, the term “conditioning” means imparting to hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, “cosmetic composition” encompasses many types of compositions for application to keratin materials such as skin or hair, for example, hair lotions, hair creams, hair gel creams, hair conditioners, hair masques (masks), etc., which can be used either as leave-on or rinse-off treatments or products.

As used herein, the term “curly hair” refers to any hair including a curl or wave. The curl may be natural or unnatural, i.e., formed by chemical treatment or physical treatment of the hair. The degree of curliness of the hair may vary and is not limited.

As used herein, the term “high heat” when used in association with hair styling refers to a temperature that is higher than about 425° F., such as from an iron, blow dryer, or the like.

As used herein, the term “high humidity” refers to conditions having at least 70% relative humidity or greater, such as at least 75% relative humidity or greater, or at least 80% relative humidity or greater.

As used herein, the term “inorganic” means a material that does not comprise carbon.

As used herein, the term “organic” means a material that is produced substantially without or essentially without the use of synthetic materials.

As used herein, the term “salts” refers to throughout the disclosure may include salts having a counter-ion such as an alkali metal, alkaline earth metal, or ammonium counterion. This list of counterions, however, is non-limiting. Salts also include a dissociated form of a compound, e.g. in an aqueous solution.

As used herein, the term “styling” the hair is intended to include “shaping,” the hair or altering the shape of hair, such as, for example, elongating hair curls or providing curl definition.

As used herein, the terms “elongating” and “stretching” hair are interchangeable, and both refer to reducing the degree of waviness or curliness and/or tightness of waves or curls of the hair, and “improving the elongation of” hair or hair curls, and variants thereof, means that the length of the hair appears closer to the actual, uncurled length of the hair. Similarly, measuring or evaluating the “elongation” of curly hair refers to the length of hair retained over a period of time, where hair having a greater elongation or length retention correspondingly has a longer appearance over such time period.

As used herein, the terms “substantially free” or “essentially free” mean the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The terms “free,” “substantially free,” and “essentially free” as used herein may also mean that the specific material is not added to the composition but may still be present in a raw material that is included in the composition. For example, a composition according to the disclosure may not include an added wax but may include a pigment that is coated with a wax and still be considered “free,” “substantially free,” or “essentially free” of waxes.

As used herein, the term “surfactants,” as well as any specifically identified surfactants, includes salts of the surfactants even if not explicitly stated.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the disclosure onto the surface of keratin materials, such as hair.

As used herein, the terms “non-sulfate-based” or “non-sulfate” anionic surfactants mean that the surfactant does not comprise a sulfate group.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts in each composition are expressed in percentage by weight (wt %) of active materials, unless otherwise defined, relative to the total weight of the composition.

### Example 1—Pre-Phase Mixtures of Citric Acid and Urea Compounds

The 5-gram pre-phase mixtures of citric acid and hydroxyethyl urea in Table 1 were prepared by mixing the specified amounts (in grams) of citric acid with aqueous hydroxyethyl urea (45% active) at room temperature.

TABLE 1

| | Inventive Pre-Phase Mixtures | | | |
|---|---|---|---|---|
| | 1A | 1B | 1C | 1D |
| Citric acid | 2.76 | 1.90 | 2.40 | 3.24 |
| Hydroxyethyl urea | 1.01 | 1.39 | 1.17 | 0.79 |
| Water | 1.23 | 1.70 | 1.43 | 0.97 |
| Total | 5 | 5 | 5 | 5 |
| Mole ratio* | 1:0.67 | 1:1.35 | 1:0.9 | 1:0.45 |
| Weight ratio* | 1:0.37 | 1:0.73 | 1:0.49 | 1:0.24 |

*Mole and weight ratios are citric acid:hydroxyethyl urea.

### Example 2—Inventive and Comparative Compositions

Inventive composition 2A and comparative compositions C1, C2, and C3, having the formulations set forth in Table 2, were prepared.

TABLE 2

| | Inventive | Comparative | | |
|---|---|---|---|---|
| | Composition | Compositions | | |
| INCI Name | 2A | C1 | C2 | C3 |
| INVENTIVE PRE-PHASE | 5 | | | |
| MIXTURE 1B (TABLE 1) | | | | |
| CITRIC ACID | | | 1.9 | |
| HYDROXYETHYL UREA | | | | 1.4 |
| COCOS NUCIFERA | 5 | 5 | 5 | 5 |
| (COCONUT) | | | | |
| OIL/COCOS NUCIFERA OIL | | | | |
| DIMETHICONE | 3 | 3 | 3 | 3 |
| CAPRYLIC/CAPRIC | 18 | 18 | 18 | 18 |
| TRIGLYCERIDE | | | | |
| BUTYLENE GLYCOL | 2.5 | 2.5 | 2.5 | 2.5 |
| C12-15 ALKYL BENZOATE | 2 | 2 | 2 | 2 |
| GLYCERIN | 4 | 4 | 4 | 4 |
| POLYACRYLAMIDE (and) | 1 | 1 | 1 | 1 |
| C13-14 ISOPARAFFIN (and) | | | | |
| LAURETH-7 | | | | |
| STEARIC ACID | 1.6 | 1.6 | 1.6 | 1.6 |
| MYRISTIC ACID | 0.09 | 0.09 | 0.09 | 0.09 |
| PALMITIC ACID | 1.4 | 1.4 | 1.4 | 1.4 |
| PROPYLENE GLYCOL | 0.04 | 0.04 | 0.04 | 0.04 |
| ETHYLHEXYLGLYCERIN | 0.1 | 0.1 | 0.1 | 0.1 |
| STEARYL ALCOHOL | 4 | 4 | 4 | 4 |
| STEARETH-100/STEARETH-2/ | 1 | 1 | 1 | 1 |
| XANTHAN GUM/MANNAN | | | | |
| ADDITIVES (fragrance, | <2 | <2 | <2 | <2 |
| dyes/pigments, plant extracts, | | | | |
| hydrating agents, preservatives, | | | | |
| pH adjusters) | | | | |
| WATER | QS to 100 | QS to | QS to | QS to |
| | | 100 | 100 | 100 |

Composition C1 was used as a base composition for preparing compositions 2A, C2, and C3. Inventive composition 2A was prepared by mixing 5% of the inventive mixture 1B from Table 1 with 95% of composition C1. Comparative composition C2 was prepared by mixing 1.4% of citric acid with 98.6% of composition C1. Comparative composition C3 was prepared by mixing 1.9% of aqueous hydroxyethyl urea (45% active) with 98.1% of composition C1.

Each of compositions 2A, C1, C2, and C3 has a pH in the range of about 3.2 to 4.2, and a viscosity ranging from about 3600 to about 6900 cPs when measured at 25° C. using a viscometer at 20 RPM with spindle 4.

### Example 3—Inventive and Comparative Compositions

Inventive composition 3A and comparative compositions C4, C5, and C6, having the formulations set forth in Table 3, were prepared.

TABLE 3

| | Inventive | Comparative | | |
|---|---|---|---|---|
| | Composition | Compositions | | |
| INCI Name | 3A | C4 | C5 | C6 |
| INVENTIVE PRE-PHASE | 5 | | | |
| MIXTURE 1B (TABLE 1) | | | | |
| CITRIC ACID | | | 1.9 | |
| HYDROXYETHYL UREA | | | | 1.4 |
| COCOS NUCIFERA | 5 | 5 | 5 | 5 |
| (COCONUT) OIL | | | | |
| BEHENTRIMONIUM | 4.0 | 4.0 | 4.0 | 4.0 |
| CHLORIDE | | | | |
| BIS-CETEARYL | 0.4 | 0.4 | 0.4 | 0.4 |
| AMODIMETHICONE | | | | |
| ISONONYL | 2.5 | 2.5 | 2.5 | 2.5 |
| ISONONANOATE | | | | |
| CETYL ESTERS | 1.5 | 1.5 | 1.5 | 1.5 |
| CETYL ESTERS | 1 | 1 | 1 | 1 |
| ISOPROPYL ALCOHOL | 0.9 | 0.9 | 0.9 | 0.9 |
| CETEARYL ALCOHOL | 5 | 5 | 5 | 5 |
| CETEARETH-25 | 0.02 | 0.02 | 0.02 | 0.02 |
| CETEARETH-7 | 0.03 | 0.03 | 0.03 | 0.03 |
| GLYCOLIC ACID | 0.002 | 0.002 | 0.002 | 0.002 |
| HYDROXY- | 0.4 | 0.4 | 0.4 | 0.4 |
| ETHYLCELLULOSE | | | | |
| CAPRYLYL GLYCOL | 0.005 | 0.005 | 0.005 | 0.005 |
| ADDITIVES (fragrance, | <2 | <2 | <2 | <2 |
| plant extracts, vitamins, pH | | | | |
| adjusters) | | | | |
| WATER | QS to 100 | QS to | QS to | QS to |
| | | 100 | 100 | 100 |

Composition C4 was used as a base composition for preparing compositions 3A, C5, and C6. Inventive composition 3A was prepared by mixing 5% of the inventive mixture 1B from Table 1 with 95% of composition C4. Comparative composition C5 was prepared by mixing 1.9% of citric acid with 98.1% of composition C4. Comparative composition C6 was prepared by mixing 1.4% of aqueous hydroxyethyl urea (45% active) with 98.6% of composition C4.

Each of compositions 3A, C4, C5, and C6 has a pH in the range of about 3 to 4, and a viscosity ranging from about 450-1100 cPs when measured using spindle 3.

### Example 4—Evaluation of Synergy of Citric Acid and Hydroxyethyl Urea

Comparative studies were conducted to evaluate the synergy of citric acid and urea compounds in hair styling/treating compositions.

### Example 4A

A comparative test was conducted to evaluate the synergistic benefits provided by the combination of citric acid and hydroxyethyl urea as included in composition 2A according to the disclosure, in comparison with comparative compositions C1-C3 of Table 2.

Before application of compositions, five swatches of curly hair of the same type were prepared by shampooing with a conventional shampoo, conditioning with a conventional conditioner, and rinsing with water. Each of compositions 2A, C1, C2, and C3 was respectively applied to one of the prepared hair swatches, in an amount of about 0.1 gram of composition per gram of a hair swatch and evenly distributed on the hair. The compositions were allowed to stay on the hair and air dried overnight at room temperature. A control swatch did not have any composition applied after the shampoo/conditioner/rinsing steps. Approximately 24 hours after the application of the compositions, the control and treated hair swatches were evaluated and images were taken.

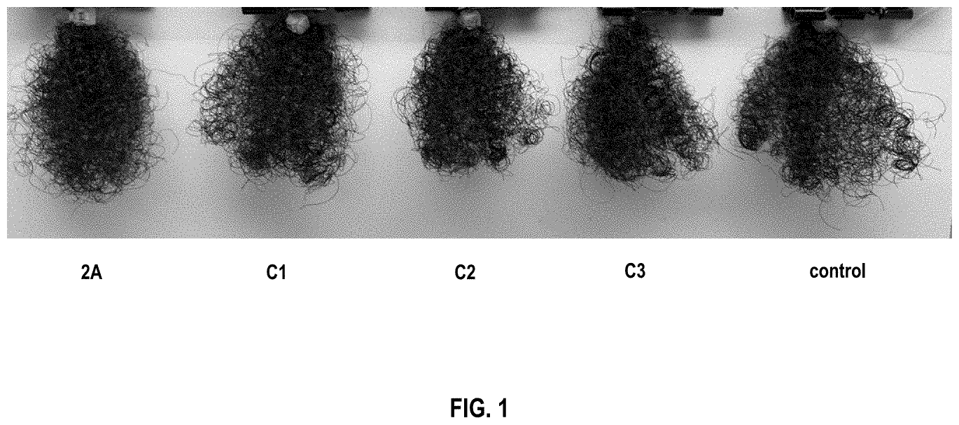

FIG. 1 shows the images of these five hair swatches 24 hours after the treatment. As shown in FIG. 1, the hair treated with inventive composition 2A shows visibly improved curl definition and regularity, as well as curl retention, where the curls are tighter and held better, compared to the untreated control hair and the hair treated with comparative compositions C1, C2, or C3. In addition, compared to the hair treated with compositions C1, C2, or C3, the hair treated with composition 2A has visibly improved frizz control, discipline (e.g., no “fly-aways”), manageability, and shaping, as well as better volume.

### Example 4B

A comparative test was conducted to evaluate the synergistic benefits provided by the combination of citric acid and hydroxyethyl urea as included in composition 3A according to the disclosure, in comparison with comparative compositions C4-C6 of Table 3.

Before application of compositions, four swatches of curly hair of the same type were shampooed with a conventional shampoo, conditioned with a conventional conditioner, and rinsed with water. Each of compositions 3A, C4, C5, and C6 was respectively applied to one of the prepared hair swatches, in an amount of about 0.1 gram of composition per gram of a hair swatch and evenly distributed on the hair. The compositions were allowed to stay on the hair and air dried overnight at room temperature. Approximately 24 hours after the application of the compositions, the control and treated hair swatches were evaluated and images were taken.

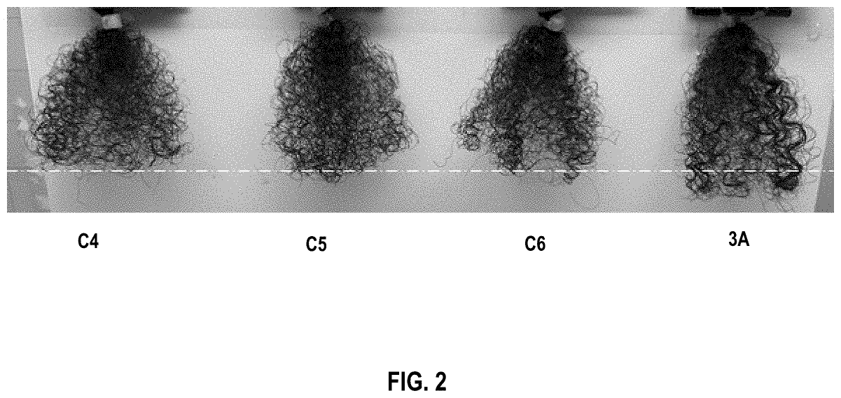

FIG. 2. shows the images of these hair swatches at 24 hours after the application of the compositions. As shown in FIG. 2, the hair treated with inventive composition 3A surprisingly show improved curl definition and elongation, as well as overall better curl properties, compared to the hair treated with comparative compositions C4, C5, or C6. The hair treated with composition 3A is visibly longer, has visibly better curl retention and tighter curl, and improved smoothness, frizz control, manageability, discipline (e.g., no “fly-aways”), and shaping compared to the hair similarly treated with compositions C4, C5, or C6.

Examples 4A and 4B thus demonstrate that the synergistic combination of citric acid and hydroxyethyl urea with a specific weight ratio, when combined with additional ingredients for styling and/or treating keratin fibers, for example, a silicone compound, fatty compounds, vegetable oils, cationic surfactants, polyols, and at an acidic pH, surprisingly and unexpected provides significantly improved curl definition and elongation, as well as other attributes such as curl hold, smoothness, frizz control, and manageability to the curly hair.

### Example 5—Hair Styling Composition

The following hair styling composition, which may optionally be used as a leave-in composition, was prepared as shown in Table 4.

TABLE 4
Inventive Composition 5A

| | |
|---|---|
| BIS-DIGLYCERYL POLYACYLADIPATE-2 | 2 |
| HYDROXYETHYL UREA | 0.7 |
| SORBITAN OLEATE | 0.003 |
| POLYQUATERNIUM-37 | 0.1 |
| ACRYLATES/STEARYL METHACRYLATE | 0.004 |
| COPOLYMER | |
| CETYL ESTERS | 0.7 |
| SORBITOL | 5 |
| GLYCINE | 0.05 |
| SUCROSE | 0.05 |
| SODIUM CITRATE | 0.3 |
| CETEARYL ALCOHOL | 4.5 |
| PPG-1 TRIDECETH-6 | 0.02 |
| PROPYLENE GLYCOL | 0.07 |
| DICAPRYLATE/DICAPRATE | |
| BEHENTRIMONIUM CHLORIDE | 2.4 |
| CITRIC ACID | 0.2 |
| PECTIN | 0.2 |
| POLYQUATERNIUM-4 | 0.3 |
| PLANT OILS (camelina sativa seed oil, | 3.1 |
| sunflower seed oil, avocado oil) | |
| ADDITIVES (fragrance, vitamins, | <3 |
| preservatives, plant extracts) | |
| SOLVENTS (water/non-aqueous solvents) | QS to 100 |

The pH of the composition was 4.0. Hair treated with composition 5A as a leave-in styling composition was visibly longer, had visibly better curl retention and tighter curl, and improved smoothness, frizz control, manageability, and discipline.

### Example 6—Additional Hair Styling Compositions

The following hair styling compositions, which are expected to provide the same hair styling benefits as in Example 5, can be prepared as shown in Table 5.

TABLE 5

| | Additional Compositions | | | |
|---|---|---|---|---|
| | 6A | 6B | 6C | 6D |
| UREA | | 1 | | 2.5 |
| DIMETHYL UREA | | | 1.4 | |
| HYDROXYETHYL UREA | 0.5 | 1 | | |
| POTASSIUM CITRATE | 0.1 | 0.1 | | 0.9 |
| CITRIC ACID | 0.2 | 0.9 | 1.4 | 1.5 |
| CETYL ESTERS | 0.5 | | | 0.5 |
| ISOPROPYL MYRISTATE | 0.5 | 1.5 | 1 | |
| GLYCERINE | 1 | 3 | 3 | |
| HYALURONIC ACID | 1.5 | | 0.5 | |
| HYDROXYETHYLCELLULOSE | 1 | 3 | 1 | 2 |
| STEARYL ALCOHOL | 3 | | 1 | |
| LAURYL ALCOHOL | 3 | 4 | | 2 |
| CETRIMONIUM CHLORIDE | 2 | 3 | 6 | 5 |
| PLANT OILS | 1 | 2 | 4 | 3 |
| ADDITIVES (e.g. fragrance, vitamins, | <5 | <5 | <5 | <5 |
| preservatives, stabilizers, emulsifiers) | | | | |
| SOLVENTS (water and optionally non- | QS to | QS to | QS to | QS to |
| aqueous solvents) | 100 | 100 | 100 | 100 |

The above examples demonstrate the surprising and unexpected synergy between citric acid and urea compounds, in particular for styling curly hair.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-48. (canceled)

49. A hair styling composition comprising:
(a) (i) citric acid, and
(ii) at least one urea compound chosen from urea and/or derivatives thereof;
(b) optionally, at least one silicone compound;
(c) at least one fatty compound other than silicone compounds; and
(d) at least one solvent,
wherein the weight ratio of the (a)(i) citric acid to the total amount of (a)(ii) urea compounds ranges from about 0.1 to about 5, and
wherein the pH of the composition is less than about 7.

50. The hair styling composition of claim 49, wherein the composition has a mole ratio of (a)(i) citric acid to the (a)(ii) urea compounds ranging from about 0.1 to about 5.

51. The hair styling composition of claim 49, wherein the total amount of (a)(i) citric acid ranges from about 0.1% to about 10% by weight, relative to the total weight of the composition.

52. The hair styling composition of claim 49, comprising (a)(ii) at least one urea compound chosen from urea, dimethyl urea, hydroxylethyl urea, or mixtures of two or more thereof.

53. The hair styling composition of claim 49, wherein the composition comprises a total amount of (a)(ii) urea compounds ranging from about 0.1% to about 10% by weight, relative to the total weight of the composition.

54. The hair styling composition of claim 49, wherein the weight ratio of the (a)(i) citric acid to the total amount of (a)(ii) urea compounds is less than about 1.

55. The hair styling composition of claim 54, wherein the composition is free of silicone compounds.

56. The hair styling composition of claim 49, wherein the composition comprises (c) at least one fatty compound chosen from vegetable oils, fatty alcohols, fatty esters, fatty ethers, or mixtures of two or more thereof.

57. The hair styling composition of claim 49, further comprising at least one cationic surfactant.

58. The hair styling composition of claim 49, comprising (b) at least one silicone compound, wherein the total amount of (b) silicone compounds ranges from about 0.01% to about 10% by weight, relative to the total weight of the composition.

59. The hair styling composition of claim 58, wherein the weight ratio of the (a)(i) citric acid to the total amount of (a)(ii) urea compounds ranges from about 1 to about 5.

60. The hair styling composition of claim 58, wherein the composition has a mole ratio of (a)(i) citric acid to the (a)(ii) urea compounds ranging from about 0.4:1 to about 3:1.

61. The hair styling composition of claim 60, further comprising at least one polyol.

62. The hair styling composition of claim 60, further comprising at least one compound chosen from emulsifiers, cationic surfactants, thickening agents, hydrating agents, or combinations of two or more thereof.

63. The hair styling composition of claim 60, wherein the composition comprises (c) at least one fatty compound chosen from vegetable oils, fatty alcohols, fatty esters, fatty ethers, or mixtures of two or more thereof.

64. A hair styling composition comprising:
(a) (i) citric acid, and
(ii) at least one urea compound chosen from urea and/or derivatives thereof;
(b) at least one non-silicone fatty compound; and
(c) water,
wherein the composition comprises from about 0.1% to about 3% of citric acid,
wherein the total amount of (a)(ii) urea compounds in the composition ranges from about 0.1% to about 3%,
wherein the weight ratio of the (a)(i) citric acid to the total amount of (a)(ii) urea compounds ranges from about 0.1 to about 1,
wherein the pH of the composition is less than about 7, and
wherein all amounts are by weight, relative to the total weight of the composition.

65. The hair styling composition of claim 64, wherein the composition comprises (b) at least one fatty compound chosen from vegetable oils, fatty alcohols, fatty esters, or combinations of two or more thereof.

66. The hair styling composition of claim 65, comprising (a)(ii) hydroxyethyl urea.

67. The hair styling composition of claim 66, wherein the composition further comprises at least one compound chosen from emulsifiers, cationic surfactants, thickening agents, hydrating agents, vitamins, or combinations of two or more thereof.

68. A method for styling hair comprising applying to the hair a composition comprising:
(a) (i) citric acid, and
(ii) at least one urea compound chosen from urea and/or derivatives thereof;
(b) optionally, at least one silicone compound;
(c) at least one fatty compound other than silicone compounds; and
(d) at least one solvent,
wherein the weight ratio of the (a)(i) citric acid to the total amount of (a)(ii) urea compounds ranges from about 0.1 to about 5, and
wherein the pH of the composition is less than about 7.
